(19) United States
(12) Patent Application Publication
(10) Pub. No.: US 2022/0220559 A1
(43) Pub. Date: Jul. 14, 2022

(54) CELL-FREE MIRNA BIOMARKERS FOR PROGNOSIS AND DIAGNOSIS OF NEURODEGENERATIVE DISEASES

(71) Applicants: Yeda Research And Development Co. Ltd., Rehovot (IL); Queen Mary University London, London (GB); UCL Business Ltd, London (GB)

(72) Inventors: Eran HORNSTEIN, Rehovot (IL); Iddo MAGEN, Rehovot (IL); Andrea MALASPINA, London (GB); Pietro FRATTA, London (GB)

(73) Assignees: Yeda Research And Development Co. Ltd., Rehovot (IL); Queen Mary University London, London (GB); UCL Business Ltd, London (GB)

(21) Appl. No.: 17/611,149

(22) PCT Filed: May 13, 2020
(86) PCT No.: PCT/IL2020/050523

§ 371 Date: Nov. 14, 2021

Related U.S. Application Data
(60) Provisional application No. 62/846,776, filed on May 13, 2019.

(51) Int. Cl. C12Q 1/6883; G01N 33/50; G01N 33/68; A61K 31/428; A61K 31/4152; A61P 25/28

(52) U.S. Cl. CPC C12Q 1/6883; G01N 33/5023; G01N 33/6896; A61K 31/428; A61K 31/4152; G01N 2333/52; C12Q 2600/178; C12Q 2600/118; C12Q 2600/112; G01N 2800/2814; A61P 25/28

(57) ABSTRACT

A method of prognosing a course of disease progression and/or survival time in a subject diagnosed with ALS or FTD is disclosed. The method comprising: (a) detecting a level of miR-181 in a biological sample of the subject; and (b) determining the disease progression and/or survival time based on the level of the miR-181, wherein: (i) when the level of miR-181 is higher than that in a control sample, it is indicative of a rapid disease progression and/or poor survival; or (ii) when the level of miR-181 is about the same or lower than that in the control sample, it is indicative of a slow disease progression and/or good survival. Methods of prognosing a stage of disease in a subject diagnosed with ALS or FTD are also disclosed.

### RELATED APPLICATION/S

This application claims the benefit of priority of U.S. Provisional Patent Application No. 62/846,776 filed on May 13, 2019, the contents of which are incorporated herein by reference in their entirety.

### FIELD AND BACKGROUND OF THE INVENTION

The present invention, in some embodiments thereof, relates to methods of diagnosing and prognosing the neurodegenerative diseases Amyotrophic lateral sclerosis (ALS) and Frontotemporal dementia (FTD) using cell-free miRNAs.

ALS is a devastating neurodegenerative syndrome of the human motor neuron system, for which no curative treatment exists. ALS is diagnosed based on medical history, clinical examination, electrophysiological findings, and exclusion of mimicking syndromes.

FTD is the most common form of dementia for people under the age of 65. It represents a group of brain disorders caused by degeneration of the frontal and/or temporal lobes of the brain. FTD represents a subgroup within the broad spectrum of neurological disorders that constitute Frontototemporal lobar degeneration (FTLD).

There has been growing evidence that FTD signs can be seen in patients primarily diagnosed with ALS, implying clinical overlap among these two disorders. Some forms of ALS and FTD share clinical, neuropathological and genetic properties and a substantial fraction of patients exhibit symptoms of both diseases. Therefore, FTD and ALS reside on clinical, neuropathological and genetic continuum [Raffaele Ferrari et al., Curr Alzheimer Res. 2011 May; 8(3): 273-294].

Reliable biomarkers from easily accessible biofluids would be of significant value for ALS and FTD diagnostics, assessment of disease progression and for disease stratification in clinical trials. In addition, biomarkers have the potential to serve as surrogate endpoints in clinical trials and to contribute to screening of asymptomatic individuals with relevant disease genetics. Cell-free ALS biomarkers include neurofilaments [Lu et al., Neurology (2015) 84(22): 2247-57] and pro-inflammatory cytokines [Prado et al., J Neurol Sci (2018) 394: 69-74]. However, both cytokines [Prado et al., 2018, supra] and neurofilament levels [Lu et al., 2015, supra] are stable over the disease course and thus may not be appropriate to track progression.

miRNAs, a class of small, non-coding RNAs, play an important role in ALS pathogenesis [Eitan and Hornstein, Brain Res (2016) 1647:105-111; and Rinchetti et al., Molecular Neurobiology (2018) 55:2617-2630]. It was previously shown that miRNAs are essential for motor neuron survival [Haramati et al., Proc Natl Acad Sci USA (2010) 107(29): 13111-6], and that miRNAs are globally downregulated in motor neurons of ALS patients [Emde et al., The EMBO journal (2015) 34(21): 2633-51]. Plasma miRNAs have been investigated as diagnostic biomarkers for ALS [Takahashi et al., Mol Brain (2015) 8(1): 67; de Andrade et al., J Neurol Sci (2016) 368: 19-24; and Sheinerman et al., Alzheimers Res Ther (2017) 9(1): 89], using microarray or qRT-PCR, which are biased towards specific miRNAs. Changes in muscle-specific miR-206, miR-1, miR-133a/b and miR-27a have been reproducibly reported [de Andrade et al., 2016, supra; and Sheinerman et al., 2017, supra; Tasca et al., Clin Neuropathol (2016) 35(1): 22-30], and changes in miR-4649-5p and miR-4299 have also been described (Takahashi et al., 2015).

Additional Background Art Includes:

U.S. Patent Application No. 20190093167 discloses methods for diagnosis and differentiation of neurodegenerative diseases by quantifying miRNAs pairs in bodily fluids.

U.S. Patent Application No. 20150164891 discloses methods of diagnosing motor neuron disease (MND) by analyzing in a sample of a subject (i) a total miR expression; and (ii) total pre-miR expression, wherein a down-regulation in (i) or (i)/(ii) beyond a predetermined threshold is indicative of the MND.

### SUMMARY OF THE INVENTION

According to an aspect of some embodiments of the present invention there is provided a method of prognosing a course of disease progression and/or survival time in a subject diagnosed with Amyotrophic lateral sclerosis (ALS) or Frontotemporal dementia (FTD), the method comprising:
(a) detecting a level of miR-181 in a biological sample of the subject; and
(b) determining the disease progression and/or survival time based on the level of the miR-181, wherein:
   (i) when the level of miR-181 is higher than that in a control sample, it is indicative of a rapid disease progression and/or poor survival; or
   (ii) when the level of miR-181 is about the same or lower than that in the control sample, it is indicative of a slow disease progression and/or good survival, thereby prognosing the course of disease progression and/or survival time.

According to an aspect of some embodiments of the present invention there is provided a method of treating ALS or FTD in a subject in need thereof, the method comprising:
(a) prognosing the subject according to the method of some embodiments of the invention; and
(b) treating the subject based on the prognosis, wherein:
   (i) when the prognosis indicates a rapid disease progression and/or poor survival, the subject is treated with an effective amount of a drug or a medicament for the treatment of rapid progressing disease; or
   (ii) when the prognosis indicates a slow disease progression and/or good survival, the subject is treated with an effective amount of a drug or a medicament for the treatment of slow progressing disease, thereby treating the ALS or FTD in the subject.

According to an aspect of some embodiments of the present invention there is provided a method of prognosing a stage of disease in a subject diagnosed with ALS or FTD, the method comprising:
(a) detecting at least two times in a course of the disease a level of miRNA of at least one of miR-423, miR-484 and miR-92 in a biological sample of the subject; and
(b) determining a stage of the disease based on the level of miRNA, wherein an increase in the level of miRNA over the at least two times in the course of the disease is indicative of progression of the disease, thereby prognosing the stage of disease.

According to an aspect of some embodiments of the present invention there is provided a method of prognosing a stage of disease in a subject diagnosed with ALS or FTD, the method comprising:
(a) detecting at least two times in a course of the disease a level of miRNA of at least one of miR-29, miR-146, miR-148 or miR-191 in a biological sample of the subject; and
(b) determining a stage of the disease based on the level of miRNA, wherein a decrease in the level of miRNA over the at least two times in the course of the disease is indicative of progression of the disease, thereby prognosing the stage of disease.

According to an aspect of some embodiments of the present invention there is provided a method of treating ALS or FTD in a subject in need thereof, the method comprising:
(a) prognosing the subject according to the method of some embodiments of the invention; and
(b) treating the subject based on the prognosis, wherein:
   (i) when the prognosis indicates an early stage of disease, the subject is treated with at least one of an effective amount of drug or medicament and/or an assistive device;
   (ii) when the prognosis indicates a middle stage of disease, the subject is treated with at least one of an effective amount of a drug or medicament, a physical therapy, an assistive device, a feeding tube, and/or a noninvasive ventilation; or
   (iii) when the prognosis indicates a late stage of disease, the subject is treated with at least one of an effective amount of a drug or medicament, a physical therapy, an assistive device, a feeding tube, and/or a noninvasive or invasive ventilation, thereby treating the ALS or FTD in the subject.

According to an aspect of some embodiments of the present invention there is provided a method of monitoring treatment in a subject diagnosed with ALS or FTD, the method comprising:
(a) treating a subject diagnosed with ALS or FTD with a drug or a medicament;
(b) detecting a level of miRNA of at least one of miR-423, miR-484, miR-92, miR-29, miR-146, miR-148 or miR-191 in a biological sample of the subject prior to and following the treatment; and
(c) determining an effective treatment based on the level of miRNA, wherein:
   (i) when the level of the miR-423, miR-484 and/or miR-92 is about the same or lower than that in a sample of the subject prior the treatment, it is indicative of an effective treatment; or
   (ii) when the level of the miR-29, miR-146, miR-148 and/or miR-191 is about the same or higher than that in a sample of the subject prior the treatment, it is indicative of an effective treatment; thereby monitoring treatment of the drug or the medicament for the treatment of ALS or FTD.

According to an aspect of some embodiments of the present invention there is provided a method for selecting subjects for enrollment in a clinical trial involving treatment of ALS or FTD, the method comprising:
(a) detecting a level of miR-181 in a biological sample of a subject diagnosed with ALS or FTD;
(b) determining the disease progression and/or survival time based on the level of the miR-181, wherein:
   (i) when the level of miR-181 is higher than that in a control sample, it is indicative of a rapid disease progression and/or poor survival; or
   (ii) when the level of miR-181 is about the same or lower than that in the control sample, it is indicative of a slow disease progression and/or good survival; and
(c) identifying the subject as being suitable for the clinical trial based on the criteria of the clinical trial.

According to an aspect of some embodiments of the present invention there is provided a method for selecting subjects for enrollment in a clinical trial involving treatment of ALS or FTD, the method comprising:
(a) detecting at least two times in a course of a disease a level of miRNA of at least one of miR-423, miR-484 and miR-92 in a biological sample of a subject diagnosed with ALS or FTD;
(b) determining a stage of the disease based on the level of miRNA, wherein an increase in the level of miRNA over the at least two times in the course of the disease is indicative of progression of the disease; and
(c) identifying the subject as being suitable for the clinical trial based on the criteria of the clinical trial.

According to an aspect of some embodiments of the present invention there is provided a method for selecting subjects for enrollment in a clinical trial involving treatment of ALS or FTD, the method comprising:
(a) detecting at least two times in a course of a disease a level of miRNA of at least one of miR-29, miR-146, miR-148 or miR-191 in a biological sample of a subject diagnosed with ALS or FTD;
(b) determining a stage of the disease based on the level of miRNA, wherein a decrease in the level of miRNA over the at least two times in the course of the disease is indicative of progression of the disease; and
(c) identifying the subject as being suitable for the clinical trial based on the criteria of the clinical trial.

According to some embodiments of the invention, one of the at least two times in the course of the disease comprises a biological sample obtained at disease onset or at time of diagnosis.

According to some embodiments of the invention, the drug comprises Riluzole, or Edavarone.

According to some embodiments of the invention, the method further comprises collecting the biological sample from the subject.

According to some embodiments of the invention, the biological sample is cell-free.

According to some embodiments of the invention, the biological sample is selected from the group consisting of a plasma, a serum and a cerebrospinal fluid sample.

According to some embodiments of the invention, the miRNA is a cell-free miRNA.

According to some embodiments of the invention, the miR-181 is a cell-free miR-181.

According to some embodiments of the invention, the detecting is effected by real time PCR (RT-PCR).

According to some embodiments of the invention, the detecting is effected by next generation sequencing (NGS).

According to some embodiments of the invention, the higher level of the miR-181 is by at least about 50%.

According to some embodiments of the invention, the lower level of the miR-181 is by about 5-30%.

According to some embodiments of the invention, the miR-181 is miR-181a-5p.

According to some embodiments of the invention, the miR-181 is miR-181b-5p.

According to some embodiments of the invention, the determining does not comprise a ratio of the miR-181 to a second miRNA selected from the group consisting of let-7e, miR-7, miR-9, miR-9*, miR-16, miR-29a, miR-31, miR-99b, miR-125b, miR-128a, miR-129-3p, miR-138, miR-155, miR-204, miR-218, miR-323-3p, miR-335, miR-338-3p, miR-451, miR-491 and miR-874.

According to some embodiments of the invention, the increase in the level of the miRNA is by at least about 50%.

According to some embodiments of the invention, the miR-423 is miR-423-5p.

According to some embodiments of the invention, the miR-92 is miR-92a-3p.

According to some embodiments of the invention, the miR-92 is miR-92b-3p.

According to some embodiments of the invention, the decrease in the level of the miRNA is by at least 50%.

According to some embodiments of the invention, the miR-29 is miR-29a-3p.

According to some embodiments of the invention, the miR-146 is miR-146b-5p.

According to some embodiments of the invention, the miR-148 is miR-148b-3p.

According to some embodiments of the invention, the miR-191 is miR-191-5p.

According to some embodiments of the invention, the determining does not comprise a ratio of the miR-29 to a second miRNA selected from the group consisting of miR-7, miR-9*, miR-99b, miR-181a, miR-206 and miR-335.

According to some embodiments of the invention, the method further comprises assessing a level of a neurofilament light chain (NfL) in the biological sample.

According to some embodiments of the invention, the method further comprises assessing a level of at least one pro-inflammatory cytokine in the biological sample.

According to some embodiments of the invention, the subject is a human being.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-C illustrate the differential miRNA expression in ALS plasma. (FIG. 1A) MA plot of differential miRNA expression in ALS (n=116 plasma samples) and healthy controls (n=105 plasma samples). Log-2 transformed fold-change, against the mean miRNA abundance. Red-significantly changed miRNAs (p-value≤0.05). (FIG. 1B) Box plot of normalized miR-206 counts for control and ALS. (FIG. 1C) receiver operating characteristic (ROC) curve of miR-206 used for distinguishing between control vs. ALS. True positive rates (sensitivity) as a function false positive rates (1-specificity) for different cut-off values. P-values are calculated given null hypothesis of area under the curve (AUC)=0.5.

FIGS. 2A-K illustrate the prognostic value of miR-181a-5p in predicting ALS patient survival from baseline and disease progression. Plots depicting correlation between survival time from baseline (first blood sampling) and the levels of: (FIG. 2A) miR-181a-5p, (FIG. 2B) miR-181b-5p, (FIG. 2C) miR-423-5p, and (FIG. 2D) miR-484. Distinct cumulative survival curves for binned baseline levels of (FIG. 2E) miR-181a-5p, (FIG. 2F) miR-181b-5p, (FIG. 2G) miR-423-5p, and (FIG. 2H) miR-484 in ALS patients. Highest 75% (red) and lowest 25% (blue) quantiles. (FIGS. 2I-K) Comparison of ALSFRS-score at baseline (FIG. 2I), disease duration at baseline (FIG. 2J) and age at baseline (FIG. 2K) between the highest 75% quantile (red) and the lowest 25% quantile (blue) of miR-181a-5p levels.

FIGS. 3A-B illustrate (FIG. 3A) Cox regression analyses for mortality hazard ratio, with sex, onset site, Riluzole treatment, progression rate at baseline (PRB) and miRNA expression levels at baseline as covariates. Survival length is calculated from baseline plasma sampling. Vertical dashed line denotes unchanged mortality (hazard ratio=1). Mean±95% CIs. (FIG. 3B) Percentage of patients with slow progression rate (≤0.5 drop in ALSFRS-R score/month from disease onset to baseline) and intermediate and fast progression rate (>0.5 drop), among the lowest 25% and highest 75% quantiles. **p<0.01, ***p<0.001, Wald test. F=females, M=males, B=bulbar, NB=non-bulbar, PRB=progression rate at baseline, H/L=highest/lowest quantiles.

FIGS. 4A-J illustrate longitudinal changes in miR-423-5p and miR-484 in ALS. (FIGS. 4A-C) MA plots of differential miRNA expression in multiple visits (t₂, t₃ and t₄), compared to baseline (t₁). Red features denote miRNAs that has changed significantly (p<0.05). (FIG. 4D) Bar graph of miR-423-5p and miR-484 levels in multiple visits, compared to baseline. (FIGS. 4E-F) plots of longitudinal changes in miR-423-5p and miR-484 levels in individual patients. (FIGS. 4G-H) Temporal changes in the levels of miR-423-5p (FIG. 4G) or miR-484 (FIG. 4H). (FIGS. 4I-J) ROC curves for (FIG. 4I) miR-423-5p and (FIG. 4J) miR-484 classifying t₄ vs. t₁.

FIGS. 5A-D illustrate that NfL and TNF-alpha correlate with miR-423-5p levels. Plasma NfL (FIG. 5A) or TNF-α (FIG. 5B) levels in ALS vs. non-neurodegeneration and miR-423-5p correlation to NfL (FIG. 5C) or TNF-α (FIG. 5D) levels. ***p<0.001, unpaired t-test.

FIGS. 6A-E illustrate the prognostic value of miR-181a-5p in predicting ALS patient survival from disease onset. Distinct cumulative survival curves for binned baseline levels of (FIG. 6A) miR-181a-5p, (FIG. 6B) miR-181b-5p (FIG. 6C) miR-423-5p, and (FIG. 6D) miR-484 in ALS patients. Highest 75% (red) and lowest 25% (blue) quantiles. (FIG. 6E) Cox regression analyses for mortality hazard ratio with sex, onset site, Riluzole treatment, and miRNA expression levels at baseline as covariates. Survival length is calculated from disease onset. Vertical dashed line denotes unchanged mortality (hazard ratio=1). Mean±95% CIs. *p<0.05, Wald test. F=females, M=males, B=bulbar, NB=non-bulbar, H/L=highest/lowest quantiles.

FIGS. 7A-C illustrate an assessment of miRNAs signal and noise in longitudinal samples from Patients with ALS. (FIG. 7A) The x-axis denotes the normalized change in miRNA levels between first and last measurements (as log 2-transformed t₄/t₁ ratios), relative to the average change of all 179 sequenced miRNAs. The y-axis denotes the variability in measurements, per-miRNA for 179 sequenced species between the 22 individuals (−log 2-transformed values of the standard error of t₄/t₁ ratios). Green, red features: are above or below the threshold of stability, respectively (−0.2). Blue features: stable miRNAs that increased progressively with disease in individual patients >1.5 SD (miR-423/484/92a/b) or <−1.5 SD (miR-29/146/148/191) from the average miRNA. (FIGS. 7B-C) Spaghetti plots of individual trajectories over four longitudinal sample collections for (FIG. 7B) miR-181a-5p and (FIG. 7C) miR-1-3p.

FIGS. 8A-C illustrate the prognostic value of miR-181a-5p in predicting ALS patient survival. (FIG. 8A) Survival predictive value of individual miRNA levels in plasma, binned by lower quantile vs. higher 3 quantiles (log rank test, y-axis), for miRNAs depicted in FIGS. 7A-C as ‘stable’. Data plotted against miRNA level correlation to survival length (absolute value of Spearman correlation-coefficient, x-axis). Cumulative survival (Kaplan-Meier) curves from enrolment (FIG. 8B) or from reported first symptoms for patients (FIG. 8C), subdivided by miR-181a-5p levels, at low quartile (30 patients) compared to the rest of the cases (85 patients).

FIGS. 9A-B illustrate the mortality hazard ratio for miR-181a-5p. Mortality hazard ratio calculated by survival length from enrolment (FIG. 9A), or from disease onset (FIG. 9B). Cox-regression analyses of covariates: miR-181a-5p levels, enrolment progression rate, enrolment ALSFRS-R score, age at onset, Riluzole-treatment, Bulbar onset, sex (Female/Male). Light blue dashed-line denotes unchanged mortality. Mean±95% CIs.

FIGS. 10A-C illustrate the prognostic value of miR-181a-5p is replicated in an independent cohort and by an orthogonal measuring technique. Kaplan-Meier curves from enrolment (FIG. 10A), or from reported first symptoms for a replication cohort (FIG. 10B), subdivided by miR-181a-5p levels, at low (14 patients) vs. high (42 patients) quantiles. (FIG. 10C) Scatter plot of plasma miR-181a-5p levels at low vs. high quantiles.

FIGS. 11A-C illustrate MA plots of differential miRNA expression upon repeated sampling relative to the first phlebotomy. Red features denote miRNAs with statistically significant change in levels.

FIGS. 12A-L illustrate the longitudinal changes in miRNAs in ALS. Temporal changes in the levels of (FIG. 12A) miR-423-5p, (FIG. 12B) miR-484, (FIG. 12C) miR-92a-3p, or (FIG. 12D) miR-92b-3p, and (FIGS. 12E-H) corresponding spaghetti plots of individual trajectories (t₁-t₄). Time intervals: t₁-t₂ 6.3±0.3 m.; t₁-t₃ 13.0±0.3 m.; t₁=t₄ 32.7±3 m. Disease duration: t₁ 28.8±3 m.; t₄ 61.5±3 m. Replication study of (FIG. 12I) miR-423-5p, (FIG. 12J) miR-484, (FIG. 12K) miR-92a-3p, and (FIG. 12L) miR-92b-3p levels by qPCR at second/first phlebotomy (t₁-t₂) in individual patients. Mean±SEM. *p<0.05, **p<0.01, ***p<0.001, one-tailed paired t-test or Wald test.

FIGS. 13A-D illustrate the longitudinal increase in miRNAs with disease progression. Correlation between the relative disease covered (rD50) in longitudinal plasma collections (X-axis) and levels of (FIG. 13A) miR-423-5p, (FIG. 13B) miR-484, (FIG. 13C) miR-92a-3p, and (FIG. 13D) miR-92b-3p.

FIGS. 14A-B illustrate plots depicting inverse correlation between miR-181a-5p and survival from first phlebotomy (FIG. 14A), or from disease onset (FIG. 14B). Of note, when the three samples taken from subjects whose survival from onset was above 260 months, Spearman correlation slightly decreases (from −0.27 to −0.25) and the p-values marginally increases (from 0.003 to 0.008), suggesting the overall data and interpretation remain unchanged.

FIGS. 15A-C illustrate that miR-181a-5p levels are not associated with disease phase. (FIG. 15A) miR-181a-5p in different phases of disease defined by rD50 values. One-way ANOVA: F (2,112)=1.06, p=0.35. (FIG. 15B) correlation of miR-181a-5p levels with rD50 (FIG. 15C) rD50 differences between low and high miR-181a-5p expression bins.

FIGS. 16A-F illustrate the correlation of miR-181a-5p levels at enrolment with phenotypic properties, including (FIG. 16A) progression rate at enrolment, (FIG. 16B) enrolment ALSFRS-score, and (FIG. 16C) patient age at disease onset. Differences in these properties between low and high miR-181a bins (FIGS. 16D-F). *p<0.05 high vs low, Mann-Whitney U test.

FIGS. 17A-E illustrate the prognostic value of miR-181 with NfL for ALS patient survival. (FIG. 17A) Kaplan-Meier (KM) survival curves: lower miR-181 quartile (30 patients) vs. the rest of the cases (85 patients). Comparable, when studied from onset (not shown). (FIG. 17B) Mortality hazard ratio by Cox-regression on miR-181 levels and covariates: enrolment progression rate, ALSFRS-R, age at onset, Riluzole therapy, onset site. Blue line—unchanged hazard ratio. Mean±95% CI. (FIG. 17C) KM curves by low/mid/high NfL tertiles (FIG. 17D) and when NfL tertiles further stratified by miR-181 levels. (FIG. 17E) Mortality hazard ratio (Cox-regression) on miR-181 levels in the middle NfL tertile.

### DESCRIPTION OF SPECIFIC EMBODIMENTS OF THE INVENTION

The present invention, in some embodiments thereof, relates to methods of diagnosing and prognosing the neurodegenerative diseases ALS and FTD using cell-free miRNAs.

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Amyotrophic lateral sclerosis (ALS) is a devastating neurodegenerative disease of the human motor neuron system, for which no curative treatment exists. Frontotemporal dementia (FTD) is the most common form of dementia for people under the age of 65. ALS and FTD are both heterogeneous at the clinical, neuropathological and genetic levels and even though they are distinct progressive disorders, there is increasing evidence that they share clinical, neuropathological and genetic features. Furthermore, early diagnosis and determination of prognosis of both neurological diseases can have a big influence on the patients, their families and assessment of therapeutic options.

While reducing the present invention to practice, the present inventors have uncovered new cell-free miRNA biomarkers for the prognosis and diagnosis ALS and FTD. These can be further utilized for determining treatment and setting criteria for inclusion in clinical trials.

As is shown hereinbelow and in the Examples section which follows, the present inventors have uncovered through laborious experimentation that miRNAs remain mostly unchanged longitudinally during ALS disease course. Utilizing a small RNA next generation sequencing in a large cohort of ALS patients with various progression rates and longitudinal samples it was uncovered that miR-423/484/92a/b increase over 30 months of disease, and miR-29/146/148/191 decrease over 30 months of disease (see Examples 4 and 6 herein below). The present inventors further uncovered that elevated miR-181a/b-5p levels predict shortened survival in two separate ALS cohorts (see Examples 2, 3 and 7 herein below). Intriguingly, the median survival of patients with low miR-181a/b-5p plasma levels was more than doubled compared to patients with high miR-181a-5p. An eight-fold difference in miR-181a-5p levels between the two prognosis subgroups was measured. Although the tissue source of miR-181a/b-5p is unknown, it emerges as a prognostic ALS biomarker that is able to predict survival and improve patient stratification in clinical trials. Moreover, it was uncovered that miR-181a/b-5p can be further utilized with other known markers of ALS, e.g. NfL (see Examples 5 and 8 herein below) and cytokine expression profiles (see Example 5 herein below) to diagnose ALS and assess disease outcome. Taken together, these observations establish a novel method of diagnosing and prognosing ALS in cell free biological samples.

Thus, according to one aspect of the present invention there is provided a method of prognosing a course of disease progression and/or survival time in a subject diagnosed with Amyotrophic lateral sclerosis (ALS) or Frontotemporal dementia (FTD), the method comprising:
(a) detecting a level of miR-181 in a biological sample of the subject; and
(b) determining the disease progression and/or survival time based on the level of the miR-181, wherein:
(i) when the level of miR-181 is higher than that in a control sample, it is indicative of a rapid disease progression and/or poor survival; or
(ii) when the level of miR-181 is about the same or lower than that in the control sample, it is indicative of a slow disease progression and/or good survival, thereby prognosing the course of disease progression and/or survival time.

According to another aspect of the present invention there is provided a method of prognosing a stage of disease in a subject diagnosed with ALS or FTD, the method comprising:
(a) detecting at least two times in a course of the disease a level of miRNA of at least one of miR-423, miR-484 and miR-92 in a biological sample of the subject; and
(b) determining a stage of the disease based on the level of miRNA, wherein an increase in the level of miRNA over the at least two times in the course of the disease is indicative of progression of the disease, thereby prognosing the stage of disease.

According to another aspect of the present invention there is provided a method of prognosing a stage of disease in a subject diagnosed with ALS or FTD, the method comprising:
(a) detecting at least two times in a course of the disease a level of miRNA of at least one of miR-29, miR-146, miR-148 or miR-191 in a biological sample of the subject; and
(b) determining a stage of the disease based on the level of miRNA, wherein a decrease in the level of miRNA over at least two times in the course of the disease is indicative of progression of the disease, thereby prognosing the stage of disease.

The term “Amyotrophic lateral sclerosis” or “ALS”, also known as Lou Gehrig's disease and Motor Neuron Disease (MND), refers to the fatal progressive neurodegenerative disease, characterized by the predominant loss of motor neurons (MNs) in primary motor cortex, the brainstem, and the spinal cord. The loss of motor neurons typically causes muscle weakness and atrophy throughout the body as both the upper and lower motor neurons degenerate, ceasing to send messages to muscles. Unable to function, the muscles gradually weaken, develop fasciculations (twitches) because of denervation, and eventually atrophy because of that denervation. Affected subjects eventually lose the ability to initiate and control all voluntary movement; bladder and bowel sphincters and the muscles responsible for eye movement are usually, but not always, spared. Cognitive or behavioral dysfunction is also associated with the disease; about half of ALS subjects experience mild changes in cognition and behavior, and 10-15% show signs of frontotemporal dementia (FTD, further discussed below). Language dysfunction, executive dysfunction, and troubles with social cognition and verbal memory are the most commonly reported cognitive symptoms in ALS.

The term “ALS”, as used herein, includes all of the classifications of ALS known in the art, including, but not limited to classical ALS (typically affecting both lower and upper motor neurons), Primary Lateral Sclerosis (PLS, typically affecting only the upper motor neurons), Progressive Bulbar Palsy (PBP or Bulbar Onset, a version of ALS that typically begins with difficulties swallowing, chewing and speaking) and Progressive Muscular Atrophy (PMA, typically affecting only the lower motor neurons).

Two forms of ALS have been described: the most common is sporadic ALS (sALS), which accounts for 90 to 95% of all cases diagnosed, the other is familial ALS (fALS), which occurs in a family lineage mainly with a dominant inheritance and accounts for about 5 to 10% of all cases. sALS and fALS are clinically indistinguishable.

MND diagnosis may be effected using gold-standard methods as well as by analyzing the levels of disease typical markers such as neurofilament light chain (NfL) and pro-inflammatory cytokines (e.g. TNF-α).

Gold standard methods include those that make up the El Escorial criteria (see, for example, Brooks et al., Amyothoph. Lateral Scler. other Motor Neuron Disorders (2000) 293-299). According to the El Escorial World Federation of Neurology, criteria for the diagnosis of ALS requires the presence of: (1) Signs of lower motor neuron (LMN) degeneration by clinical, electrophysiological or neuropathologic examination; (2) Signs of upper motor neuron (UMN) degeneration by clinical examination, and (3) Progressive spread of signs within a region or to other regions, together with the absence of: Electrophysiological evidence of other disease processes that might explain the signs of LMN and/or UMN degenerations; and Neuroimaging evidence of other disease processes that might explain the observed clinical and electrophysiological signs.

Other diagnostic methods that can be used for diagnosis of ALS are those that involve transcranial magnetic stimulation (TMS). This noninvasive procedure creates a magnetic pulse inside the brain that stimulates motor activity in a certain area of the body. Electrodes taped to different areas of the body pick up and record the electrical activity in the muscles.

Below is a list of such tests:

1. Electromyography (EMG) is used to diagnose muscle and nerve dysfunction and spinal cord disease. It is also used to measure the speed at which impulses travel along a particular nerve. EMG records the electrical activity from the brain and/or spinal cord to a peripheral nerve root (found in the arms and legs) that controls muscles during contraction and at rest. Very fine wire electrodes are inserted one at a time into a muscle to assess changes in electrical voltage that occur during movement and when the muscle is at rest. The electrodes are attached to a recording instrument. Testing usually lasts about an hour or more, depending on the number of muscles and nerves to be tested.

2. EMG is usually done in conjunction with a nerve conduction velocity study. This procedure also measures electrical energy to test the nerve's ability to send a signal. A technician tapes two sets of flat electrodes on the skin over the muscles. The first set of electrodes is used to send small pulses of electricity (similar to a jolt from static electricity) to stimulate the nerve that directs a particular muscle. The second set of electrodes transmits the responding electrical signal to a recording machine. The physician then reviews the response to verify any nerve damage or muscle disease.

3. Laboratory screening tests of blood, urine, or other substances can rule out muscle diseases and other disorders that may have symptoms similar to those of MND. For example, analysis of the fluid that surrounds the brain and spinal cord can detect a number of disorders, including PPS. Blood tests may be ordered to measure levels of the protein creatine kinase (which is needed for the chemical reactions that produce energy for muscle contractions); high levels may help diagnose muscle diseases such as muscular dystrophy.

4. Magnetic resonance imaging (MRI) uses computer-generated radio waves and a powerful magnetic field to produce detailed images of body structures including tissues, organs, bones, and nerves. These images can help diagnose brain and spinal cord tumors, eye disease, inflammation, infection, and vascular irregularities that may lead to stroke. MRI can also detect and monitor degenerative disorders such as multiple sclerosis and can document brain injury from trauma. MRI is often used to rule out diseases other than the MNDs that affect the head, neck, and spinal cord.

5. Muscle or nerve biopsy can help confirm nerve disease and nerve regeneration. A small sample of the muscle or nerve is removed under local anesthetic and studied under a microscope. The sample may be removed either surgically, through a slit made in the skin, or by needle biopsy, in which a thin hollow needle is inserted through the skin and into the muscle. A small piece of muscle remains in the hollow needle when it is removed from the body. Although this test can provide valuable information about the degree of damage, it is an invasive procedure that may itself cause neuropathic side effects. Many experts do not believe that a biopsy is always needed for diagnosis.

ALS is typically divided into three stages (also referred to as “stage of disease”) based on disease symptoms. In the early stages of ALS (preliminary stage) signs of any one of e.g. muscle weakness, muscle switching (fasciculation), muscle cramping, fatigue, poor balance and slurred speech may be evident. In the middle stages of ALS (moderate disease), more severe symptoms are typically evident, e.g. severe muscle weakness, paralysis in some muscles, difficulty in swallowing/eating/chewing, breathing difficulties and/or pseudobulbar affect. In the late stages of ALS (late stage disease), severe symptoms are typically evident, e.g. paralysis in most muscles, extremely limited mobility, inability to speak, inability to breath/eat/drink (i.e. without assistance).

Method of monitoring ALS progression are well known in the art. Non-limiting examples of such methods include Physical evaluation by a physician; Weight; Electrocardiogram (ECG); ALS Functional Rating Scale (ALSFRS or ALSFRS-R) score; respiratory function which can be measured by e.g. vital capacity (forced vital capacity or slow vital capacity); muscle strength which can be measured by e.g. hand held dynamometry (HHD), hand grip strength dynamometry, manual muscle testing (MMT), electrical impedance myography (EIM) and Maximum Voluntary Isometric Contraction Testing (MVICT); motor unit number estimation (MUNE); cognitive/behavior function which can be measured by e.g. the ALS Depression Inventory (ADI-12), the Beck Depression Inventory (BDI) and the Hospital Anxiety Depression Scale (HADS) questionnaires; Quality of life which can be evaluated by e.g. the ALS Assessment Questionnaire (ALSAQ-40); and Akt level, Akt phosphorylation and/or pAkt:tAkt ratio (see International Patent Application Publication No. WO2012/160563, the contents of which are fully incorporated herein by reference).

The test for generating the ALSFRS-R score includes 12 questions that relate to the ability to perform a task. Patients are rated on their ability to perform a task on a point scale (e.g., a five-point scale from 0=can't do, to 4=normal ability). Individual item scores are summed to produce a total score (e.g., 0=worst and 48=best). The ALSFRS-R model can predict an ALSFRS-R score at a defined time in the future (for example, in 6 months, in 1 year, in 5 years, etc.) for a patient based on clinical data for the patient.

The term “Frontotemporal dementia” or “FTD”, is a neurodegenerative disease characterized by severe frontotemporal lobar degeneration. FTD is distinguished from Alzheimer's disease and Lewy body dementia based on several factors one of which being that it does not manifest with amyloid plaques, neurofibrillary tangles, or Lewy bodies. Symptoms of FTD typically appear around 45 to 65 years of age, regardless of gender. Symptoms typically progress at a rapid, steady rate. Some patients with FTD also exhibit motor neuron disease (MND) e.g. ALS (i.e. FTD-ALS).

FTD is difficult to diagnose due to the heterogeneity of the associated symptoms. Symptoms are classified into three groups based on the functions of the frontal and temporal lobes: (1) Behavioral variant FTD (bvFTD) exhibits symptoms of lethargy and aspontaneity on the one hand, and disinhibition on the other. Apathetic patients may become socially withdrawn and stay in bed all day or no longer take care of themselves. Disinhibited patients can make inappropriate (sometimes sexual) comments or perform inappropriate acts (e.g. stealing or speeding). (2) Progressive nonfluent aphasia (PNFA), also referred to as nonfluent variant primary progressive aphasia (nfvPPA), presents with a breakdown in speech fluency due to articulation difficulty, phonological and/or syntactic errors but preservation of word comprehension. (3) Semantic dementia (SD), also referred to as semantic variant primary progressive aphasia (svPPA), can be found in some patients that remain fluent with normal phonology and syntax, but increasing difficulty with naming and word comprehension. It has been researched that some may even go through depression and lose their inhibitions and exhibit antisocial behavior.

FTD diagnosis may be effected using the criteria proposed by the international consortium in 2011. A summary of these criteria can be found in Bott et al., Neurodegener Dis Manag. (2014) 4(6): 439-454, incorporated herein by reference in its entirety.

In short, diagnosis of bvFTD typically requires a patient to have a progressive deterioration of behavior accompanied by three out of six core features (disinhibition, apathy, loss of sympathy/empathy, eating behavior changes, compulsive behaviors and an executive predominant pattern of dysfunction on cognitive testing). Additionally, functional decline and neuroimaging consistent with bvFTD are used for diagnosis. Neuroimaging findings include e.g. frontal, or anterior temporal atrophy, or both, on CT or MRI, or frontal hypoperfusion or hypometabolism on single-photon emission computed tomography (SPECT) or PET. Clinical syndrome may be further supported by genetic or pathological confirmation.

With respect to nfvPPA, diagnosis typically requires either agrammatism in language production or effortful, halting speech with inconsistent speech sound errors and distortions (AOS), along with two of the three remaining core features (impaired comprehension of syntactically complex sentences, spared single-word comprehension and spared object knowledge). In addition, neuroimaging consistent with nfvPPA supports diagnosis, and typically shows either predominant left posterior fronto-insular atrophy on MRI, or predominant left posterior fronto-insular hypoperfusion or hypometabolism on SPECT or PET, or both. Clinical syndrome may be further supported by genetic or pathological confirmation.

With respect to svPPA, diagnosis typically requires both impaired confrontation naming, and single-word comprehension, with at least 3 out of 4 additional core features (impaired object knowledge, surface dyslexia or dysgraphia, spared repetition and spared speech production). In addition, neuroimaging consistent with svPPA supports diagnosis, and typically shows either predominant anterior temporal lobe atrophy, or predominant anterior temporal hypoperfusion or hypometabolism on SPECT or PET, or both. Clinical syndrome may be further supported by genetic or pathological confirmation.

As used herein, the term “subject” refers to an animal, preferably a mammal, most preferably a human being of any gender or age (e.g., infant, child or adult) who has been diagnosed with ALS or FTD, or is predisposed to ALS or FTD. The subject of some embodiments may show preliminary signs of ALS, may have a moderate ALS disease, or may have a full blown late stage ALS disease. The subject of some embodiments may have behavioral variant FTD (bvFTD), a semantic variant primary progressive aphasia (svPPA), or a nonfluent variant primary progressive aphasia (nfvPPA). Moreover, the subject of some embodiments may have FTD-ALS. Alternatively, the subject may have a genetic predisposition to the ALS, FTD or FTD-ALS.

The term “diagnosed”, “diagnosis” or “diagnosing” as used herein refers to methods by which the skilled artisan can estimate and/or determine whether or not a patient is suffering from a given disease or condition (e.g. ALS or FTD). The skilled artisan often makes a diagnosis on the basis of one or more diagnostic indicators, e.g., a marker, the presence, absence, amount, or change in amount of which is indicative of the presence, severity, or absence of the condition. Such diagnostic methods are discussed above.

As used herein the term “prognosed”, “prognosis” or “prognosing” refers to the prediction of a likely course or outcome of a disease or disease progression, particularly with respect to a likelihood of, for example, recurrence, remission, relapse, and death (i.e., the outlook for chances of survival or time remaining for survival).

As used herein, “good prognosis” or “favorable prognosis” means a likelihood that an individual having the disease will remain with mild symptoms for a prolonged period of time or that the symptoms will occur gradually over time (e.g. slow decline of muscle function and/or cognition and/or psychiatric/behavior symptoms). Good prognosis is typically associated with a slow disease progression and good survival time (i.e. prolonged survival).

As used herein, “poor prognosis” means a likelihood that an individual having the disease will exhibit severe symptoms in a short period of time (e.g. rapid decline of muscle function and/or cognition and/or psychiatric/behavior symptoms). These symptoms may continue to progress (e.g. leading to death), or may plateau and remain stable over a period of time. Poor prognosis is typically associated with a rapid disease progression and poor survival time (i.e. shorter duration).

As used herein, the phrase “rapid progression” or “rapid progressing disease” refers to a disease (e.g. ALS) in which the symptoms progress continuously and significant degradation of motor neurons can be observed within less than a year with subject survival of up to 4 years from diagnosis. According to specific embodiments, the rapid progression ALS is characterized by a change of above 1.0 ALSFRS-R points over a period of 1 month (i.e. calculated as the change of ALSFRS-R score divided by disease duration from onset in months).

As used herein, the phrase “slow progression” or “slow progressing disease” refers to a disease (e.g. ALS) in which the symptoms progress gradually and degradation of motor neurons can be observed over a period of a few years with subject survival of more than 5 years from diagnosis. According to specific embodiments, the slow progression ALS is characterized by a change of below 0.5 ALSFRS-R points over a period of 1 month (i.e. calculated as the change of ALSFRS-R score divided by disease duration from onset in months).

According to one embodiment, individuals having a slow progressing ALS or good prognosis are expected to survive at least 5 years after diagnosis. Such individuals may survive 6 years, 7 years, 10 years or more after diagnosis. Conversely, individuals having a rapid prognosing ALS or poor prognosis are expected to survive no more than 4 years after diagnosis. Such individuals may survive for only 3 years, 2 years, 18 months, 12 months or even 6 months after diagnosis.

According to one embodiment, individuals having a slow progressing FTD or good prognosis are expected to survive at least 5 years after diagnosis. Such individuals may survive 8 years, 10 years, 15 years or more after diagnosis. Conversely, individuals having a rapid progressing FTD or poor prognosis are expected to survive no more than 3 years after diagnosis. Such individuals may survive for only 2.5 years, 2 years, 18 months, 12 months or even 6 months after diagnosis.

According to one embodiment, individuals having a slow progressing FTD-ALS, individuals having a slow progression or good prognosis are expected to survive at least 3 years after diagnosis. Such individuals may survive 4 years, 5 years or more after diagnosis. Conversely, individuals having a rapid progressing FTD-ALS or poor prognosis are expected to survive no more than 2 years after diagnosis. Such individuals may survive for only 18 months, 12 months or even 6 months after diagnosis.

The method of some embodiments of the invention enables prognosis of ALS or FTD based on detection of miRNAs in a biological sample of the subject.

As used herein, the term “microRNA”, “miRNA”, and “miR” are synonymous and refer to a collection of non-coding single-stranded RNA molecules of about 19-28 nucleotides in length, which regulate gene expression (acting as post-transcriptional regulators). MicroRNAs are typically processed from pre-miR (pre-microRNA precursors, typically of 45-90, 60-80 or 60-70 nucleotides). Pre-miRs are a set of precursor miRNA molecules transcribed by RNA polymerase III that are efficiently processed into functional miRNAs (i.e. mature miRNAs). According to one embodiment, this term encompasses any type of micoRNA including 5 prime (i.e. miR or 5p) or 3 prime (i.e. miR* or 3p) and their precursors.

Exemplary miRNAs and their precursors are provided below (accession numbers per miRbase).

According to one embodiment, the miRNA comprises miR-181 or a precursor thereof.

According to a specific embodiment miR-181 is a miR-181a precursor (e.g. as set forth in MI0000269 or MI0000289).

According to a specific embodiment miR-181 is miR-181a-5p (e.g. as set forth in MIMAT0000256).

According to a specific embodiment miR-181 is miR-181a-3p (e.g. miR-181a-2-3p as set forth in MIMAT0004558 or MIMAT0000270).

According to a specific embodiment miR-181 is a miR-181b precursor (e.g. as set forth in MI0000270 or MI0000683).

According to a specific embodiment miR-181 is miR-181b-5p (e.g. as set forth in MIMAT0000257).

According to a specific embodiment miR-181 is miR-181b-3p (e.g. as set forth in MIMAT0022692 or MIMAT0031893).

According to a specific embodiment miR-181 is a miR-181c precursor (e.g. as set forth in MI0000271).

According to a specific embodiment miR-181 is miR-181c-5p (e.g. as set forth in MIMAT0000258).

According to a specific embodiment miR-181 is miR-181c-3p (e.g. as set forth in MIMAT0004559).

According to a specific embodiment miR-181 is a miR-181d precursor (e.g. as set forth in MI0003139).

According to a specific embodiment miR-181 is miR-181d-5p (e.g. as set forth in MIMAT0002821).

According to a specific embodiment miR-181 is miR-181d-3p (e.g. as set forth in MIMAT0026608).

According to one embodiment, the miRNA comprises miR-423 or a precursor thereof.

According to a specific embodiment miR-423 is a miR-423 precursor (e.g. as set forth in MI0001445).

According to a specific embodiment miR-423 is miR-423-5p (e.g. as set forth in MIMAT0004748).

According to a specific embodiment miR-423 is miR-423-3p (e.g. as set forth in MIMAT0001340).

According to one embodiment, the miRNA comprises miR-484 or a precursor thereof.

According to a specific embodiment miR-484 is a miR-484 precursor (e.g. as set forth in MI0002468).

According to a specific embodiment miR-484 is a mature miR-484 (e.g. as set forth in MIMAT0002174).

According to one embodiment, the miRNA comprises miR-92 or a precursor thereof.

According to a specific embodiment miR-92 is a miR-92a precursor (e.g. as set forth in MI0000093 or MI0000094).

According to a specific embodiment miR-92 is miR-92a-5p (e.g. as set forth in MIMAT0004507 or MIMAT0004508).

According to a specific embodiment miR-92 is miR-92a-3p (e.g. as set forth in MIMAT0000092).

According to a specific embodiment miR-92 is a miR-92b precursor (e.g. as set forth in MI0003560).

According to a specific embodiment miR-92 is miR-92b-5p (e.g. as set forth in MIMAT0004792).

According to a specific embodiment miR-92 is miR-92b-3p (e.g. as set forth in MIMAT0003218).

According to one embodiment, the miRNA comprises miR-29 or a precursor thereof.

According to a specific embodiment miR-29 is a miR-29a precursor (e.g. as set forth in MI0000087).

According to a specific embodiment miR-29 is miR-29a-5p (e.g. as set forth in MIMAT0004503).

According to a specific embodiment miR-29 is miR-29a-3p (e.g. as set forth in MIMAT0000086).

According to a specific embodiment miR-29 is a miR-29b precursor (e.g. as set forth in MI0000105 or MI0000107).

According to a specific embodiment miR-29 is miR-29b-5p (e.g. as set forth in MIMAT0004514 or MIMAT0004515).

According to a specific embodiment miR-29 is miR-29b-3p (e.g. as set forth in MIMAT0000100).

According to a specific embodiment miR-29 is a miR-29c precursor (e.g. as set forth in MI0000735).

According to a specific embodiment miR-29 is miR-29c-5p (e.g. as set forth in MIMAT0004673).

According to a specific embodiment miR-29 is miR-29c-3p (e.g. as set forth in MIMAT0000681).

According to one embodiment, the miRNA comprises miR-146 or a precursor thereof.

According to a specific embodiment miR-146 is a miR-146a precursor (e.g. as set forth in MI0000477).

According to a specific embodiment miR-146 is miR-146a-5p (e.g. as set forth in MIMAT0000449).

According to a specific embodiment miR-146 is miR-146a-3p (e.g. as set forth in MIMAT0004608).

According to a specific embodiment miR-146 is a miR-146b precursor (e.g. as set forth in MI0003129).

According to a specific embodiment miR-146 is miR-146b-5p (e.g. as set forth in MIMAT0002809).

According to a specific embodiment miR-146 is miR-146b-3p (e.g. as set forth in MIMAT0004766).

According to one embodiment, the miRNA comprises miR-148 or a precursor thereof.

According to a specific embodiment miR-148 is a miR-148a precursor (e.g. as set forth in MI0000253).

According to a specific embodiment miR-148 is miR-148a-5p (e.g. as set forth in MIMAT0004549).

According to a specific embodiment miR-148 is miR-148a-3p (e.g. as set forth in MIMAT0000243).

According to a specific embodiment miR-148 is a miR-148b precursor (e.g. as set forth in MI0000811).

According to a specific embodiment miR-148 is miR-148b-5p (e.g. as set forth in MIMAT0004699).

According to a specific embodiment miR-148 is miR-148b-3p (e.g. as set forth in MIMAT0000759).

According to one embodiment, the miRNA comprises miR-191 or a precursor thereof.

According to a specific embodiment miR-191 is a miR-191 precursor (e.g. as set forth in MI0000465).

According to a specific embodiment miR-191 is miR-191-5p (e.g. as set forth in MIMAT0000440).

According to a specific embodiment miR-191 is miR-191-3p (e.g. as set forth in MIMAT0001618).

According to one embodiment, the term miRNA comprises a combination of any two or more (e.g. 2, 3, 4, 5 or more) of the above described miRNAs.

As used herein “a biological sample” refers to a sample of fluid or tissue sample derived from a subject. Examples of fluid samples include, but are not limited to, blood, plasma, serum, cerebrospinal fluid (CSF), lymph fluid, tears, saliva, sputum, urine and semen. An example of a tissue sample includes a brain tissue sample or a nerve tissue sample (e.g. for post-mortem diagnosis).

According to a specific embodiment, the biological sample is cell-free.

According to a specific embodiment, the biological sample comprises cell-free miRNA.

Typically, a biological sample is obtained from a subject (e.g. blood or CSF) and cells are removed therefrom when needed. Cell-free samples include, but are not limited to, plasma, serum and CSF.

Procedures for obtaining biological samples (e.g., blood samples or CSF samples) from individuals are well known in the art. Such procedures include, but are not limited to, standard blood retrieval procedures, lumbar puncture and urine collection. These and other procedures for obtaining biological samples are described in details in www(dot)healthatoz(dot)com/healthatoz/Atoz/searchdotasp.

Regardless of the procedure employed, once the biological sample is obtained, the level of miRNA (e.g. cell-free miRNA) in the biological sample is determined.

As used herein the phrase “cell-free miRNA” refers to miRNA present within the cell-free fraction of a biological sample. The cell-free miRNA described herein is not comprised in intact cells (i.e., comprising uncompromised plasma membrane) but may be associated with cell-derived vesicles (e.g. exosomes).

Cell-free miRNA may be extracted from the biological sample according to any method known in the art. For instance, after obtaining the biological sample (i.e. blood or CSF), all nucleated cells are removed from the sample by two centrifugation cycles (e.g. at 1,600×g for 10 minutes at 4° C.). Total RNA is extracted from the cell-free sample (e.g. plasma or serum or CSF) using, for example, the miRNeasy micro kit (Qiagen, Hilden, Germany) and quantified with, for example, Qubit fluorometer using RNA broad range (BR) assay kit (Thermo Fisher Scientific, Waltham, Mass.).

According to one embodiment, the method further comprises collecting a biological sample from the subject.

According to one embodiment, two or more samples are collected from a subject.

According to one embodiment, biological samples are obtained at different time points in the course of the disease (e.g. at 2, 3, 4, 5 or more time points in the course of the disease). For example, samples may be collected at disease onset, at the time of disease diagnosis or at different times during disease progression (e.g. at early stages of ALS, at middle stages of ALS and/or at late stages of ALS, as discussed above).

According to one embodiment, a sample is obtained at disease onset or at diagnosis. According to one embodiment, a second, third, forth (or more) sample is obtained at least 1, 2, 3, 4, 5, 6, 7, 8, 9, 10, 11, 12, 15, 18, 21, 24, 27, 30, 36 or more months after disease onset or diagnosis. According to one embodiment, samples are obtained within 1, 2, 3, 4, 5, 6, 7, 8, 9, 10, 11, 12, 15, 18, 21, 24, 27, 30, 36 or more months from each other.

It will be appreciated that the samples can be of the same source (e.g. blood or CSF), but can also be obtained from different sources (e.g. blood and CSF), i.e. of the same subject. If a plurality of samples are analyzed at different time points, they should be from the same origin (i.e. source).

miRNA levels may be examined at each time point and compared to each other. Additionally or alternatively, miRNA levels may be examined at each time point and compared to corresponding miRNA levels of control samples (i.e. a sample obtained from an individual not diagnosed with ALS or FTD, or of an individual diagnosed with a slow disease prognosis and/or good survival). Such control samples are typically obtained from subjects of the same age and gender. Under certain circumstances it may even be derived of the same subject prior to disease diagnosis. Furthermore, normal miRNA levels may be determined experimentally or derived from the literature if available.

The expression level of the miRNA in the biological samples of some embodiments of the invention can be determined using any methods known in the arts. For example, a number of miRNA quantitative analysis methods have been developed, including e.g. miRNA chip arrays, SYBR Green I-based miRNA qRT-PCR assays [discussed in Raymond et al., Simple, quantitative primer-extension PCR assay for direct monitoring of microRNAs and short-interfering RNAs. RNA (2005) 11], stem-loop-based TaqMan assays [discussed in Chen C et al., Real-time quantification of microRNAs by stem-loop RT-PCR. Nucleic Acids Res (2005) 33(20):e179], beads-based assays, high throughput sequencing and the like.

According to one embodiment, detecting a specific miRNA (e.g. cell-free miRNA) with or without a step of amplification typically involves the use of at least one of next generation sequencing (NGS), real time PCR, nCounter (Nanostring), or microarray (as described in detail in the ‘general materials and experimental procedures’ section of the Examples section which follows).

As mentioned, prognosing a course of disease progression and/or survival in a subject diagnosed with ALS or FTD is affected by determining the level of miR-181 in a biological sample of the subject as compared to a control sample (e.g. of an individual diagnosed with a slow disease prognosis and/or good survival).

According to one embodiment, when the level of miR-181 is higher than that in a control sample (e.g. of an individual diagnosed with a slow disease prognosis and/or good survival), it is indicative of a rapid disease progression and/or poor survival.

According to one embodiment, the miR-181 level in a biological sample of a subject is higher by about 40-100% as compared to a control sample.

According to one embodiment, the miR-181 level in a biological sample of a subject is higher by at least about 40%, 50%, 60%, 70%, 80%, 90%, 100% or more, as compared to a control sample.

According to one embodiment, the miR-181 level in a biological sample of a subject is higher by at least about 40% as compared to a control sample.

According to one embodiment, the miR-181 level in a biological sample of a subject is higher by at least about 50% as compared to a control sample.

According to one embodiment, the miR-181 level in a biological sample of a subject is higher by at least about 60% as compared to a control sample.

According to one embodiment, the miR-181 level in a biological sample of a subject is higher by at least about 70% as compared to a control sample.

According to one embodiment, the miR-181 level in a biological sample of a subject is higher by at least about 80% as compared to a control sample.

According to one embodiment, the miR-181 level in a biological sample of a subject is higher by at least about 90% as compared to a control sample.

According to one embodiment, the miR-181 level in a biological sample of a subject is higher by at least about 100% as compared to a control sample.

According to one embodiment, the miR-181 level in a biological sample of a subject is higher by 1.5-10 fold or more, as compared to a control sample.

According to one embodiment, the miR-181 level in a biological sample of a subject is higher by at least about 1.5 fold, 2 fold, 3 fold, 4 fold, 5 fold, 6 fold, 7 fold, 8 fold, 9 fold, 10 fold or more, as compared to a control sample.

According to one embodiment, the miR-181 level in a biological sample of a subject is higher by at least about 2 fold as compared to a control sample.

According to one embodiment, the miR-181 level in a biological sample of a subject is higher by at least about 4 fold as compared to a control sample.

According to one embodiment, the miR-181 level in a biological sample of a subject is higher by at least about 5 fold as compared to a control sample.

According to one embodiment, the miR-181 level in a biological sample of a subject is higher by at least about 6 fold as compared to a control sample.

According to one embodiment, the miR-181 level in a biological sample of a subject is higher by at least about 7 fold as compared to a control sample.

According to one embodiment, the miR-181 level in a biological sample of a subject is higher by at least about 8 fold as compared to a control sample.

According to one embodiment, when the level of miR-181 in a biological sample of a subject is about the same or lower than that in a control sample (e.g. of an individual diagnosed with a slow disease prognosis and/or good survival), it is indicative of a slow disease progression and/or good survival.

According to one embodiment, the miR-181 level in a biological sample of a subject is higher by no more than about 1-30% as compared to a control sample.

According to one embodiment, the miR-181 level in a biological sample of a subject is higher by no more than about 30% as compared to a control sample.

According to one embodiment, the miR-181 level in a biological sample of a subject is higher by no more than about 20% as compared to a control sample.

According to one embodiment, the miR-181 level in a biological sample of a subject is higher by no more than about 10% as compared to a control sample.

According to one embodiment, the miR-181 level in a biological sample of a subject is higher by no more than about 5% as compared to a control sample.

According to one embodiment, the miR-181 level in a biological sample of a subject is lower by about 1-30% as compared to a control sample.

According to one embodiment, the miR-181 level in a biological sample of a subject is lower by about 20% as compared to a control sample.

According to one embodiment, the miR-181 level in a biological sample of a subject is lower by about 10% as compared to a control sample.

According to one embodiment, the miR-181 level in a biological sample of a subject is lower by about 5% as compared to a control sample.

According to one embodiment, the miR-181 level in a biological sample of a subject is comparable that of a control sample.

According to one embodiment, determining a course of disease progression and/or survival in a subject diagnosed with ALS or FTD does not comprise a ratio of miR-181 to a second miRNA selected from the group consisting of let-7e, miR-7, miR-9, miR-9*, miR-16, miR-29a, miR-31, miR-99b, miR-125b, miR-128a, miR-129-3p, miR-138, miR-155, miR-204, miR-218, miR-323-3p, miR-335, miR-338-3p, miR-451, miR-491 and miR-874.

As mentioned, prognosing a stage of disease in a subject diagnosed with ALS or FTD is affected by determining the level of miRNA of at least one of miR-423, miR-484, miR-92, miR-29, miR-146, miR-148 or miR-191 in samples obtained at least two times in the course of the disease (i.e. from the same subject).

As mentioned above, a sample may be obtained 1, 2, 3, 4, 5, 6, 7, 8, 9, 10, 11, 12, 15, 18, 21, 24, 27, 30, 36 or more months after disease onset or diagnosis. According to one embodiment, samples are obtained within 1, 2, 3, 4, 5, 6, 7, 8, 9, 10, 11, 12, 15, 18, 21, 24, 27, 30, 36 or more months from each other.

According to one embodiment, a first sample of the at least two samples is obtained at disease onset or at initial time of diagnosis.

According to one embodiment, 2, 3, 4, 5 or more samples are obtained from a subject for comparison.

According to one embodiment, prognosing a stage of disease in a subject diagnosed with ALS or FTD is affected by determining the miRNA level of any one of miR-423, miR-484 or miR-92 in biological samples of the subject.

According to one embodiment, prognosing a stage of disease in a subject diagnosed with ALS or FTD is affected by determining the miRNA level of any two of miR-423, miR-484 or miR-92.

According to one embodiment, prognosing a stage of disease in a subject diagnosed with ALS or FTD is affected by determining the miRNA level of miR-423, miR-484 and miR-92.

According to one embodiment, an increase in the miRNA level of at least one of miR-423, miR-484 and/or miR-92 over the at least two times in the course of the disease is indicative of progression of the disease.

According to one embodiment, the increase in miRNA level of at least one of miR-423, miR-484 and/or miR-92 is by about 10-100% in a later collected sample as compared to an earlier collected sample of the same subject.

According to one embodiment, the increase in miRNA level of at least one of miR-423, miR-484 and/or miR-92 is by at least about 10%, 20%, 30%, 40%, 50%, 60%, 70%, 80%, 90%, 100% or more, in a later collected sample as compared to an earlier collected sample of the same subject.

According to one embodiment, the increase in miRNA level of at least one of miR-423, miR-484 and/or miR-92 is by at least about 10% in a later collected sample as compared to an earlier collected sample of the same subject.

According to one embodiment, the increase in miRNA level of at least one of miR-423, miR-484 and/or miR-92 is by at least about 20% in a later collected sample as compared to an earlier collected sample of the same subject.

According to one embodiment, the increase in miRNA level of at least one of miR-423, miR-484 and/or miR-92 is by at least about 40% in a later collected sample as compared to an earlier collected sample of the same subject.

According to one embodiment, the increase in miRNA level of at least one of miR-423, miR-484 and/or miR-92 is by at least about 50% in a later collected sample as compared to an earlier collected sample of the same subject.

According to one embodiment, the increase in miRNA level of at least one of miR-423, miR-484 and/or miR-92 is by at least about 60% in a later collected sample as compared to an earlier collected sample of the same subject.

According to one embodiment, the increase in miRNA level of at least one of miR-423, miR-484 and/or miR-92 is by at least about 80% in a later collected sample as compared to an earlier collected sample of the same subject.

According to one embodiment, the increase in miRNA level of at least one of miR-423, miR-484 and/or miR-92 is by at least about 100% in a later collected sample as compared to an earlier collected sample of the same subject.

According to one embodiment, the increase in miRNA level of at least one of miR-423, miR-484 and/or miR-92 is by 1.5-10 fold or more in a later collected sample as compared to an earlier collected sample of the same subject.

According to one embodiment, the increase in miRNA level of at least one of miR-423, miR-484 and/or miR-92 is by at least about 1.5, 2, 3, 4, 5, 6, 7, 8, 9, 10 fold or more in a later collected sample as compared to an earlier collected sample of the same subject.

According to one embodiment, the increase in miRNA level of at least one of miR-423, miR-484 and/or miR-92 is by at least about 1.5 fold or more in a later collected sample as compared to an earlier collected sample of the same subject.

According to one embodiment, the increase in miRNA level of at least one of miR-423, miR-484 and/or miR-92 is by at least about 2 fold or more in a later collected sample as compared to an earlier collected sample of the same subject.

According to one embodiment, the increase in miRNA level of at least one of miR-423, miR-484 and/or miR-92 is by at least about 3 fold or more in a later collected sample as compared to an earlier collected sample of the same subject.

According to one embodiment, the increase in miRNA level of at least one of miR-423, miR-484 and/or miR-92 is by at least about 4 fold or more in a later collected sample as compared to an earlier collected sample of the same subject.

According to one embodiment, prognosing a stage of disease in a subject diagnosed with ALS or FTD is affected by determining the miRNA level of any one of miR-29, miR-146, miR-148 or miR-191 in biological samples of the subject.

According to one embodiment, prognosing a stage of disease in a subject diagnosed with ALS or FTD is affected by determining the miRNA level of any two of miR-29, miR-146, miR-148 or miR-191.

According to one embodiment, prognosing a stage of disease in a subject diagnosed with ALS or FTD is affected by determining the miRNA level of any three of miR-29, miR-146, miR-148 or miR-191.

According to one embodiment, prognosing a stage of disease in a subject diagnosed with ALS or FTD is affected by determining the miRNA level of miR-29, miR-146, miR-148 and miR-191.

According to one embodiment, a decrease in the miRNA level of at least one of miR-29, miR-146, miR-148 and miR-191 over the at least two times in the course of the disease is indicative of progression of the disease.

According to one embodiment, the decrease in miRNA level of at least one of miR-29, miR-146, miR-148 and/or miR-191 is by at least about 10-100% in a later collected sample as compared to an earlier collected sample of the same subject.

According to one embodiment, the decrease in miRNA level of at least one of miR-29, miR-146, miR-148 and/or miR-191 is by at least about 10%, 20%, 30%, 40%, 50%, 60%, 70%, 80%, 90%, 100% or more, in a later collected sample as compared to an earlier collected sample of the same subject.

According to one embodiment, the decrease in miRNA level of at least one of miR-29, miR-146, miR-148 and/or miR-191 is by at least about 10% in a later collected sample as compared to an earlier collected sample of the same subject.

According to one embodiment, the decrease in miRNA level of at least one of miR-29, miR-146, miR-148 and/or miR-191 is by at least about 20% in a later collected sample as compared to an earlier collected sample of the same subject.

According to one embodiment, the decrease in miRNA level of at least one of miR-29, miR-146, miR-148 and/or miR-191 is by at least about 40% in a later collected sample as compared to an earlier collected sample of the same subject.

According to one embodiment, the decrease in miRNA level of at least one of miR-29, miR-146, miR-148 and/or miR-191 is by at least about 50% in a later collected sample as compared to an earlier collected sample of the same subject.

According to one embodiment, the decrease in miRNA level of at least one of miR-29, miR-146, miR-148 and/or miR-191 is by at least about 60% in a later collected sample as compared to an earlier collected sample of the same subject.

According to one embodiment, the decrease in miRNA level of at least one of miR-29, miR-146, miR-148 and/or miR-191 is by at least about 80% in a later collected sample as compared to an earlier collected sample of the same subject.

According to one embodiment, the decrease in miRNA level of at least one of miR-29, miR-146, miR-148 and/or miR-191 is by at least about 100% in a later collected sample as compared to an earlier collected sample of the same subject.

According to one embodiment, the decrease in miRNA level of at least one of miR-29, miR-146, miR-148 and/or miR-191 is by 1.5-10 fold or more in a later collected sample as compared to an earlier collected sample of the same subject.

According to one embodiment, the decrease in miRNA level of at least one of miR-29, miR-146, miR-148 and/or miR-191 is by at least about 1.5, 2, 3, 4, 5, 6, 7, 8, 9, 10 fold or more in a later collected sample as compared to an earlier collected sample of the same subject.

According to one embodiment, the decrease in miRNA level of at least one of miR-29, miR-146, miR-148 and/or miR-191 is by at least about 1.5 fold or more in a later collected sample as compared to an earlier collected sample of the same subject.

According to one embodiment, the decrease in miRNA level of at least one of miR-29, miR-146, miR-148 and/or miR-191 is by at least about 2 fold or more in a later collected sample as compared to an earlier collected sample of the same subject.

According to one embodiment, the decrease in miRNA level of at least one of miR-29, miR-146, miR-148 and/or miR-191 is by at least about 3 fold or more in a later collected sample as compared to an earlier collected sample of the same subject.

According to one embodiment, the decrease in miRNA level of at least one of miR-29, miR-146, miR-148 and/or miR-191 is by at least about 4 fold or more in a later collected sample as compared to an earlier collected sample of the same subject.

According to one embodiment, determining a stage of disease in a subject diagnosed with ALS or FTD does not comprise a ratio of miR-29 to a second miRNA selected from the group consisting of miR-7, miR-9*, miR-99b, miR-181a, miR-206 and miR-335.

Prognosis of disease progression, survival time and/or stage of disease according to the present teachings may be confirmed by gold standard methods and by methods of monitoring ALS progression, as discussed in detail above.

According to some embodiments, the method further comprises assessing a level of a neurofilament light chain (NfL) in a biological sample.

Neurofilament is an axonal structural protein that is released as a result of neuroaxonal damage during neurodegeneration. Thus, following neurodegeneration, NfL can be detected in CSF and blood and serves as a diagnostic marker of ALS. However, while higher baseline NfL levels (e.g. levels above 150 pg/ml) are associated with poor survival and lower NfL levels (e.g. levels below 80 pg/ml) are associated with good survival, intermediate NfL levels (e.g. levels of 80-150 pg/ml) are not informative for diagnosis or prognosis.

According to one embodiment of the invention, the combined assessment of NfL and miR-181 is used for prognosis of disease prognosis and/or survival time.

According to one embodiment, detection of an intermediate level of NfL and detection of miR-181 levels higher than that in a control sample (as discussed above), are indicative of a rapid disease progression and/or poor survival.

According to one embodiment, detection of an intermediate level of NfL and detection of miR-181 levels about the same or lower than that in a control sample (as discussed above), are indicative of a slow disease progression and/or good survival.

According to some embodiments, the method further comprises assessing a level of at least one cytokine, growth factor, or a receptor thereof in a biological sample.

According to some embodiments, the method further comprises assessing a level of at least one pro-inflammatory cytokine in a biological sample.

According to one embodiment, the cytokine (e.g. pro-inflammatory cytokine), growth factor or receptor thereof comprise, for example, TNF-α, TNF receptor 1, IL-6, IL-1β, IL-8, and vascular endothelial growth factor (VEGF).

Any method known in the art may be employed to assess the level of cytokine (e.g. pro-inflammatory cytokine), growth factor or receptor thereof in a biological sample (e.g. blood, serum). Exemplary methods include Western Blot and ELISA.

Once prognosis is substantiated, the subject may be treated based on the results of the prognosis. Accordingly, treatment is determined based on disease stage (e.g. early, middle or late stage of ALS, or classification group for FTD), the course of disease progression (e.g. slow or rapid disease), and/or survival time (e.g. good or poor survival time).

According to some embodiments of the invention, the method further comprises informing the subject of the predicted prognosis.

As used herein the phrase “informing the subject” refers to advising the subject that based on the methods of some embodiments of the invention the subject should seek a suitable treatment regimen.

Once the prognosis is determined, the results can be recorded in the subject's medical file, which may assist in selecting a treatment regimen and/or determining prognosis of the subject.

As mentioned, the prognosis of a subject can be used to select the treatment regimen of a subject and thereby treat the subject in need thereof.

As used herein, the terms “treating” or “treatment” include abrogating, substantially inhibiting, slowing or reversing the progression of a condition, substantially ameliorating clinical or aesthetical symptoms of a condition or substantially preventing the appearance of clinical or aesthetical symptoms of a condition (e.g., ALS or FTD).

Any drug or medicament for the treatment of ALS or FTD may be used in accordance with the present teachings.

According to one embodiment, the drug or medicament is for the treatment of rapid progressing disease.

According to one embodiment, the drug or medicament is for the treatment of slow progressing disease.

According to one embodiment, the drug or medicament is a candidate drug for therapy (e.g. an experimental drug or a drug in a clinical trial).

Exemplary agents which may be used in accordance with the present teachings for the treatment of ALS or ALS symptoms include, but are not limited to, is Riluzole (e.g. Rilutek® or Tiglutik®), Edavarone (e.g. Radicava® and Radicut®), dextromethorphan and quinidine (Nuedexta®), antidepressants and anti-anxiety drugs. Additionally, gene therapy, antisense oligonucleotide therapy, and cellular base therapy (e.g. injection of mesenchymal stem cells) can be used for the treatment of ALS.

Exemplary agents which may be used in accordance with the present teachings for the treatment of FTD or FTD symptoms include, but are not limited to, drugs which are used to manage the behavioral symptoms, antidepressants, drugs for treatment of aggression, agitation and psychosis, and drugs for the treatment of dementia. Exemplary drugs for the treatment of FTD include, but are not limited to, selective serotonin reuptake inhibitors (SSRIs), anti-depressants (e.g. trazodone), neuroleptics/antipsychotics (e.g. olanzapine, risperidone and aripiprazole), cholinergic agents (e.g. rivastigmine), acetylcholinesterase inhibitors (e.g. galantamine), NMDA receptor antagonists (e.g. Memantine). Additionally, gene therapy, antisense oligonucleotide therapy, and cellular base therapy (e.g. injection of mesenchymal stem cells) can be used for the treatment of FTD.

According to one embodiment, the subject is treated with a nutraceutical composition i.e. any substance that may be considered a food or part of a food and provides medical or health benefits, including the prevention and treatment of disease. In some embodiments, a nutraceutical composition is intended to supplement the diet and contains at least one or more of the following ingredients: a vitamin; a mineral; an herb; a botanical; a fruit; a vegetable; an amino acid; or a concentrate, metabolite, constituent, or extract of any of the previously mentioned ingredients; and combinations thereof.

In some embodiments, a nutraceutical composition of the present invention can be administered as a “dietary supplement,” as defined by the U.S. Food and Drug Administration, which is a product taken by mouth that contains a “dietary ingredient” such as, but not limited to, a vitamin, a mineral, an herb or other botanical, an amino acid, and substances such as an enzyme, an organ tissue, a glandular, a metabolite, or an extract or concentrate thereof.

According to one embodiment, the subject is treated with physical therapy, or any other therapy which may assist muscle movement or pain.

According to one embodiment, the subject is treated with an assistive device. Any assistive device can be used according to the present teachings including, but not limited to, a cane, a leg brace, a hand and/or wrist splint, a wheelchair (such as a power wheelchair), a communication device, and a mechanical lift. Additionally or alternatively, any of a feeding tube, a urinary catheter, a ventilator (e.g., noninvasive such as a BiPAP e.g. by Philips Respironics) or invasive ventilator (e.g. via tracheostomy) or a pacemaker may be used.

Any of the above described agents may be administered or used individually or in combination.

According to one aspect of the invention, there is provided a method of treating ALS or FTD in a subject in need thereof, the method comprising:
(a) prognosing the subject according to the method of some embodiments of the invention; and
(b) treating the subject based on the prognosis, wherein:
   (i) when the prognosis indicates a rapid disease progression and/or poor survival, the subject is treated with an effective amount of a drug or a medicament for the treatment of rapid progressing disease; or
   (ii) when the prognosis indicates a slow disease progression and/or good survival, the subject is treated with an effective amount of a drug or a medicament for the treatment of slow progressing disease, thereby treating the ALS or FTD in the subject.

According to one aspect of the invention, there is provided a method of treating ALS or FTD in a subject in need thereof, the method comprising:
(a) prognosing the subject according to the method of some embodiments of the invention; and
(b) treating the subject based on the prognosis, wherein:
(i) when the prognosis indicates an early stage of disease, the subject is treated with at least one of an effective amount of a drug or medicament (e.g. riluzole, edaravone, antidepressant drug, anti-anxiety drug, and/or drug for treatment of pseudobulbar affect) and/or an assistive device (e.g. cane or brace);
(ii) when the prognosis indicates a middle stage of disease, the subject is treated with at least one of an effective amount of a drug or medicament (e.g. riluzole, edaravone, antidepressant drug, anti-anxiety drug, and/or drug for treatment of pseudobulbar affect), a physical therapy, an assistive device (e.g. cane, leg brace, hand and wrist splint, wheelchair, etc.), a feeding tube, and/or a noninvasive ventilation (e.g., a BiPAP e.g. by Philips Respironics); or
(iii) when the prognosis indicates a late stage of disease, the subject is treated with at least one of an effective amount of a drug or medicament (e.g. riluzole, edaravone, antidepressant drug, anti-anxiety drug, and/or drug for treatment of pseudobulbar affect), a physical therapy, an assistive device (e.g. power wheelchair, communication devices, etc.), a feeding tube, and/or a noninvasive ventilation (e.g., a BiPAP e.g. by Philips Respironics) or invasive ventilation (e.g. via tracheostomy), thereby treating the ALS or FTD in the subject.

According to one aspect of the invention, there is provided a use of a medicament for treating ALS or FTD in a subject in need thereof, comprising:
(a) prognosing the subject according to the method of some embodiments of the invention; and
(b) using a drug or a medicament for the treatment of rapid progressing disease for treating the subject when the prognosis indicates a rapid disease progression and/or poor survival; or
(c) using an effective amount of a drug or a medicament for the treatment of slow progressing disease for treating the subject when the prognosis indicates a slow disease progression and/or good survival.

According to one aspect of the invention, there is provided a use of a medicament for treating ALS or FTD in a subject in need thereof, comprising:
(a) prognosing the subject according to the method of some embodiments of the invention; and
(b) using an effective amount of at least one of a drug or medicament (e.g. riluzole, edaravone, antidepressant drug, anti-anxiety drug, and/or drug for treatment of pseudobulbar affect) or an assistive device (e.g. cane or brace) for treating the subject when the prognosis indicates an early stage of disease;
(c) using at least one of an effective amount of a drug or medicament (e.g. riluzole, edaravone, antidepressant drug, anti-anxiety drug, and/or drug for treatment of pseudobulbar affect), a physical therapy, an assistive device (e.g. cane, leg brace, hand and wrist splint, wheelchair, etc.), a feeding tube, and/or a noninvasive ventilation (e.g., a BiPAP e.g. by Philips Respironics) for treating the subject when the prognosis indicates a middle stage of disease, or
(iii) using at least one of an effective amount of a drug or medicament (e.g. riluzole, edaravone, antidepressant drug, anti-anxiety drug, and/or drug for treatment of pseudobulbar affect), a physical therapy, an assistive device (e.g. power wheelchair, communication devices, etc.), a feeding tube, and/or a noninvasive ventilation (e.g., a BiPAP e.g. by Philips Respironics) or invasive ventilation (e.g. via tracheostomy) for treating the subject when the prognosis indicates a late stage of disease.

The methods of some embodiments of the invention may be further used for monitoring treatment of ALS or FTD.

According to one aspect of the invention, there is provided a method of monitoring treatment in a subject diagnosed with ALS or FTD, the method comprising:
(a) treating a subject diagnosed with ALS or FTD with a drug or a medicament (e.g. as specified above);
(b) detecting a level of miRNA of at least one of miR-423, miR-484, miR-92, miR-29, miR-146, miR-148 or miR-191 in a biological sample of the subject prior to and following the treatment; and
(c) determining an effective treatment based on the level of miRNA, wherein:
(i) when the level of the miR-423, miR-484 and/or miR-92 is about the same or lower than that in a sample of the subject prior the treatment, it is indicative of an effective treatment; or
(ii) when the level of the miR-29, miR-146, miR-148 and/or miR-191 is about the same or higher than that in a sample of the subject prior the treatment, it is indicative of an effective treatment, thereby monitoring treatment of the drug or the medicament for the treatment of ALS or FTD.

According to one aspect of the invention, there is provided a method of monitoring treatment in a subject diagnosed with ALS or FTD, the method comprising:
(a) detecting a level of miRNA of at least one of miR-423, miR-484, miR-92, miR-29, miR-146, miR-148 or miR-191 in a biological sample of a subject having been treated with a drug or medicament (as specified above) prior to and following the treatment; and
(b) determining an effective treatment based on the level of miRNA, wherein:
   (i) when the level of the miR-423, miR-484 and/or miR-92 is about the same or lower than that in a sample of the subject prior the treatment, it is indicative of an effective treatment; or
   (ii) when the level of the miR-29, miR-146, miR-148 and/or miR-191 is about the same or higher than that in a sample of the subject prior the treatment, it is indicative of an effective treatment, thereby monitoring treatment of the drug or the medicament for the treatment of ALS or FTD.

The methods of some embodiments of the invention may be further used for selecting subjects for enrollment in a clinical trial involving treatment of ALS or FTD.

According to one aspect of the invention, there is provided a method for selecting subjects for enrollment in a clinical trial involving treatment of ALS or FTD, the method comprising:
(a) detecting a level of miR-181 in a biological sample of a subject diagnosed with ALS or FTD;
(b) determining the disease progression and/or survival time based on the level of the miR-181, wherein:
   (i) when the level of miR-181 is higher than that in a control sample, it is indicative of a rapid disease progression and/or poor survival; or
   (ii) when the level of miR-181 is about the same or lower than that in the control sample, it is indicative of a slow disease progression and/or good survival; and
(c) identifying the subject as being suitable for the clinical trial based on the criteria of the clinical trial.

According to one aspect of the invention, there is provided a method for selecting subjects for enrollment in a clinical trial involving treatment of ALS or FTD, the method comprising:
(a) detecting at least two times in a course of a disease a level of miRNA of at least one of miR-423, miR-484 and miR-92 in a biological sample of a subject diagnosed with ALS or FTD;
(b) determining a stage of the disease based on the level of miRNA, wherein an increase in the level of miRNA over the at least two times in the course of the disease is indicative of progression of the disease; and
(c) identifying the subject as being suitable for the clinical trial based on the criteria of the clinical trial.

According to one aspect of the invention, there is provided a method for selecting subjects for enrollment in a clinical trial involving treatment of ALS or FTD, the method comprising:
(a) detecting at least two times in a course of a disease a level of miRNA of at least one of miR-29, miR-146, miR-148 or miR-191 in a biological sample of a subject diagnosed with ALS or FTD;
(b) determining a stage of the disease based on the level of miRNA, wherein a decrease in the level of miRNA over the at least two times in the course of the disease is indicative of progression of the disease; and
(c) identifying the subject as being suitable for the clinical trial based on the criteria of the clinical trial.

It is expected that during the life of a patent maturing from this application many relevant drugs or medicaments for the treatment of ALS or FTD will be developed and the scope of the term drug or medicament for the treatment of ALS or FTD is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Experimental Procedures

Standard Protocol Approvals, Registrations, and Patient Consents

All participants provided written consent (or gave verbal permission for a carer to sign on their behalf) to be enrolled in the ALS biomarkers study and ethical approval was obtained from the East London and the City Research Ethics Committee 1 (09/H0703/27).

Study Design

For survival analysis, 120 ALS patients were needed to obtain a hazard ratio of 3-fold in short survivors compared with long survivors, with a power of 99% and a p-value of 0.01. The sample size was determined based on these calculations. Because sample processing was done in different batches, samples were randomly allocated to the batches except for longitudinal ALS samples from the same individual that were sequenced in the same batch to avoid batch-induced biases in interpretation of longitudinal changes. RNA-seq data from plasma at study enrolment were collected retrospectively, while phenotypic data on de-identified patients was separated and blinded.

Participants and Sampling

This study included a first cohort with 115 patients with ALS and 107 healthy controls from the ALS biomarker study. ALS patients were diagnosed according to standard criteria by experienced ALS neurologists as described in Brooks et al. [Brooks et al., Amyotroph Lateral Scler Other Motor Neuron Disord (2000) 1(5): 293-9] and were enrolled if they met inclusion criteria until the desired sample size was reached (consecutive series). Healthy controls were typically spouses or relatives of patients. Informed consent was obtained from all participants. Serial plasma samples and clinical information were obtained, on average, every 2 to 4 months from 22 of the 115 patients with ALS. No selection criteria were applied to individuals with ALS sampled longitudinally, other than their willingness to donate further samples. Symptom onset was defined as first patient-reported weakness. Disease severity was assessed with the revised ALS Functional Rating Scale (ALSFRS-R) [Cedarbaum et al., J Neurol Sci (1999) 169: 13-21], and progression rate at enrolment (i.e. first blood draw) was calculated as follows: (48-enrolment ALSFRS-R)/time (in months) from symptom onset to enrolment. Progression was also modeled using the D50 model which fits a sigmoid decay across all available ALSFRS-R scores [Prell et al., Front Aging Neurosci (2019) 11: 5; and Poesen et al., Neurology (2017) 88: 2302-2309]. Use of Riluzole (or not) at the time of sampling was recorded. In samples from cohort I (115 ALS patients), miRNAs were profiled with NGS. A second cohort of 56 ALS patients was used to validate the results of the first cohort by an orthogonal method of quantitative real time PCR. Plasma samples were stored in −80° C. until RNA extraction and subsequent small RNA next generation sequencing.

Quantification of NfL and TNF-α

Plasma samples were processed and aliquoted within 1 hour from collection and frozen at −80° C., following standard consensus procedures as previously described [Teunissen et al., Neurology (2009) 73(22): 1914-22]. Baseline Neurofilament light chain (NfL) levels were measured in plasma samples by an electrochemiluminescence immunoassay [Lu et al., 2015, supra] (Lu et al., 2015a). TNF-α was measured as previously reported [Lu et al., Neurol Neuroimmunol Neuroinflamm (2016) 3(4): e244].

Quantification of Small RNA by Next Generation Sequencing

Total RNA was extracted from plasma using the miRNeasy micro kit (Qiagen, Hilden, Germany) and quantified with Qubit fluorometer using RNA broad range (BR) assay kit (Thermo Fisher Scientific, Waltham, Mass.). For small RNA next generation sequencing (NGS), libraries were prepared from 7.5 ng of total RNA using the QIAseq™ miRNA Library Kit and QIAseq miRNA NGS 48 Index IL (Qiagen), by an experimenter who was blinded to the identity of samples. Following 3′ and 5′ adapter ligation, small RNA was reverse transcribed, using unique molecular identifier (UMI), primers of random 12-nucleotide sequences. This way, precise linear quantification miRNA was achieved, overcoming potential PCR-induced biases as previously discussed by Coenen-Stass et al. [Coenen-Stass et al., RNA Biol (2018) 15(8): 1133-45]. cDNA libraries were amplified by PCR for 22 cycles, with a 3′ primer that included a 6-nucleotide unique index. Following size selection and cleaning of libraries with magnetic beads, quality control was performed by measuring library concentration with Qubit fluorometer using dsDNA high sensitivity (HS) assay kit (Thermo Fisher Scientific, Waltham, Mass.) and confirming library size with Tapestation D1000 (Agilent). Libraries with different indices were multiplexed and sequenced on a single NextSeq 500/550 v2 flow cell (Illumina), with 75 bp single read and 6 bp index read. Fastq files were demultiplexed using the User-friendly Transcriptome Analysis Pipeline (UTAP) developed at the Weizmann Institute [Kohen et al., BMC bioinformatics (2019) 24: 154]. Sequences were mapped to the human genome using Qiagen GeneGlobe analysis web tool.

Quantitative Real Time PCR

TaqMan Advanced MicroRNA cDNA Synthesis Kit (Applied Biosystems) was used for cDNA reverse transcription (10 ng input). Universal PCR Master Mix without AmpErase UNG on white reaction plates (MicroAmp EnduraPlate Optical 96-well, Thermo Fisher) and run on a StepOnePlus machine (Applied Biosystems). TaqMan qPCR was performed using Advanced MicroRNA Assays (reaction volume 10 μL) with the following probes: hsa-miR-181a-5p (Assay ID: 477857_mir); hsa-miR-423-5p (Assay ID: 478090_mir); hsa-miR-484 (Assay ID: 478308_mir); hsa-miR-92a-3p (Assay ID: 477827_mir); hsa-miR-92b-3p (Assay ID: 477823_mir); hsa-miR-140-3p (Assay ID: 477908_mir) and hsa-miR-185-5p (Assay ID: 477939_mir). Normalizers were selected based on stable expression in the first cohort: (1) basemean expression between 500-3,000; (2) coefficient of variation ≤0.35 [Zhou et al., PLoS One (2017) 12: e0185288] and (3) minimal, insignificant, changes in patient survival between different expression bins of the miRNA. Relative miR-181a-5p quantity, normalized to the average expression of hsa-miR-140-3p and hsa-miR-185-5p, were binned to lowest 25% and highest 75% for Kaplan-Meier survival analysis. Relative miR-423/484/92a/92b quantities, normalized to same normalizers was compared between enrolment sample (t₁) and corresponding follow-up sample (t₂) by one-tailed paired t-test.

Statistical Analysis

Plasma samples with ≥50,000 total miRNA unique molecular identifiers (UMIs) were included in the analysis of prognostic biomarkers, i.e. stratification of survival lengths according to low/high expression bins of miRNAs. miRNA differential expression in next generation sequencing (NGS) data was analyzed via DESeq2 package in R Project for Statistical Computing [R Core Team. R: A Language and Environment for Statistical Computing. Vienna, Austria. R Foundation for Statistical Computing (2015)], under the assumption that miRNA counts followed negative binomial distribution. In cross-sectional analysis of differential expression (ALS vs. controls), read counts were corrected for covariates such as the subject's sex and the day in which his/her plasma sample was processed, to minimize potential confounding effects. In longitudinal analysis, sequencing reads were corrected for subject, as each subject had measurements from multiple time points. Fold-change values in miRNA abundance between ALS patients and control samples were calculated as the ratio of normalized counts in ALS to the normalized counts in the control group, and presented after logarithmic transformation on base 2. miRNAs with an average abundance of ≥50 UMIs per sample, were considered above noise levels. P values were calculated by Wald test [Anders S and Huber W, Genome Biol (2010) 11(10): R106; and Love et al., Genome Biol (2014) 15(12): 550] and then adjusted for multiple testing according to Benjamini and Hochberg [Benjamini et al., Behav Brain Res (2001) 125(1-2): 279-84]. Adjusted p values<0.05 were considered significant.

Log rank Mantel-Cox test was used to compare Kaplan-Meier survival curves, and Cox regression was used to calculate mortality hazard ratio, using age, sex, onset site, treatment status, miRNA levels (e.g. miR-181a-5p levels) and phenotypic features as covariates. For binary classification by miRNAs, receiver operating characteristic (ROC) curves for individual miRNAs or combinations of miRNAs were plotted based on voom transformation of gene expression data in R [Law et al., Genome Biol (2014) 15(2): R29]. For longitudinal miRNA expression by qPCR, one-tailed paired t-test was used. Outliers were detected by Grubbs test [Grubbs, Technometrics (1969) 11: 1-21] and excluded from analysis. Graphs were generated with GraphPad Prism 5.

### Example 1

Muscle-Enriched miRNAs are Increased in the Plasma of ALS Patients

In order to characterize the potential of plasma miRNAs as biomarkers for ALS a first cohort of 225 plasma samples was assembled and subjected to next generation sequencing. 105 of 107 control samples and 116 of the 118 ALS samples (>98% in both groups) passed the threshold of 50,000 miRNA UMIs detected and were included in the differential expression analysis. Control and ALS cohorts were comparable in terms of excluded sample fraction (p=1, Fisher's exact test). As many as 1973 individual miRNA species were aligned to the human genome (GRCh37/hg19) across all samples. However, only 215 miRNA species exceeded a cut-off of ≥50 UMIs per sample, after averaging across all samples. Individual samples expressed on average 173±2 miRNAs at expression levels of ≥50 UMIs.

TABLE 1
Summary of demographic and clinical characteristics
of ALS patients and controls

| | ALS patients | Controls |
|---|---|---|
| Sex (M/F) | 75/41** | 46/59 |
| Age at baseline, years | 63.9 ± 1.0*** | 50.6 ± 1.5 |
| El-Escorial | 34/54/14/14 | N/A |
| (Definite/probable/possible/lab-supported) | | |
| Clinical onset (bulbar/non-bulbar) | 38/78 | N/A |
| Riluzole treated/untreated/unknown | 81/34/1 | N/A |
| Sporadic/familial | 109/7 | N/A |
| C9ORF72 negative or unknown/positive | 108/8 | N/A |
| Disease duration at first sampling, months | 28.2 ± 3.4 | N/A |
| ALSFRS-R | 35.7 ± 0.8 | N/A |

Abbreviations: ALSFRS-R: ALS functional rating scale.
**Percentage of males/females significantly different from controls (Fisher’s exact test: p = 0.002).
***Significantly different from controls (t-test: p < 0.001).

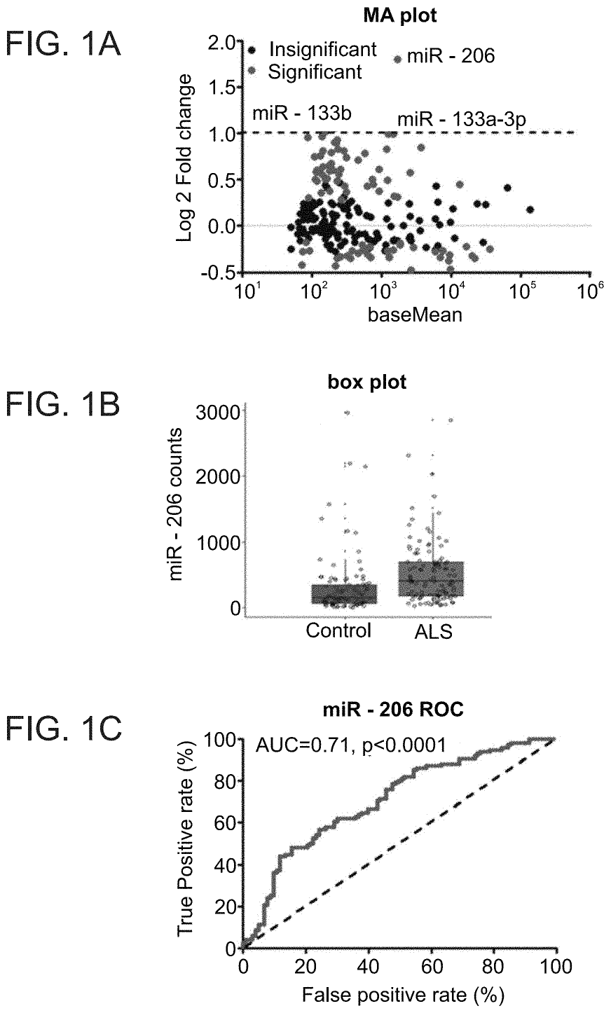

From the 216 detected miRNAs, 36 decreased in abundance and 54 increased in abundance in a statistically significant manner in ALS samples relative to controls (adjusted p-values<0.05, Wald test, FIG. 1A). Two miRNAs increased in a distinctive manner: miR-3168, which has not been previously reported, increased 3.15 fold over control plasma (adjusted p<0.0001). In addition, miR-206 was upregulated 3.5 fold, relative to control (FIG. 1B, adjusted p-value<0.0001). miR-206 is a muscle-enriched microRNA that was reported to increase in ALS patient blood [Tasca et al., 2016, supra]. Other skeletal muscle-enriched miRNAs that were reported as being elevated in ALS biofluids [Tasca et al., 2016, supra], miR-133a-3p and miR-133b, were also elevated in our cohort (2 and 2.1 fold, respectively, p=0.0007). These observations resonate with previous miRNA biomarker studies, using an independent, larger British cohort.

Receiver-operating characteristic (ROC) curves were generated in order to determine miRNA predictive power as binary disease classifiers. The area under the curve (AUC) for miR-206 ROC curve demonstrated modest predictive power: its AUC was 0.71±0.04 (FIG. 1C, p<0.001).

### Example 2

Higher miR-181a-5p Levels are Associated with Poor Survival

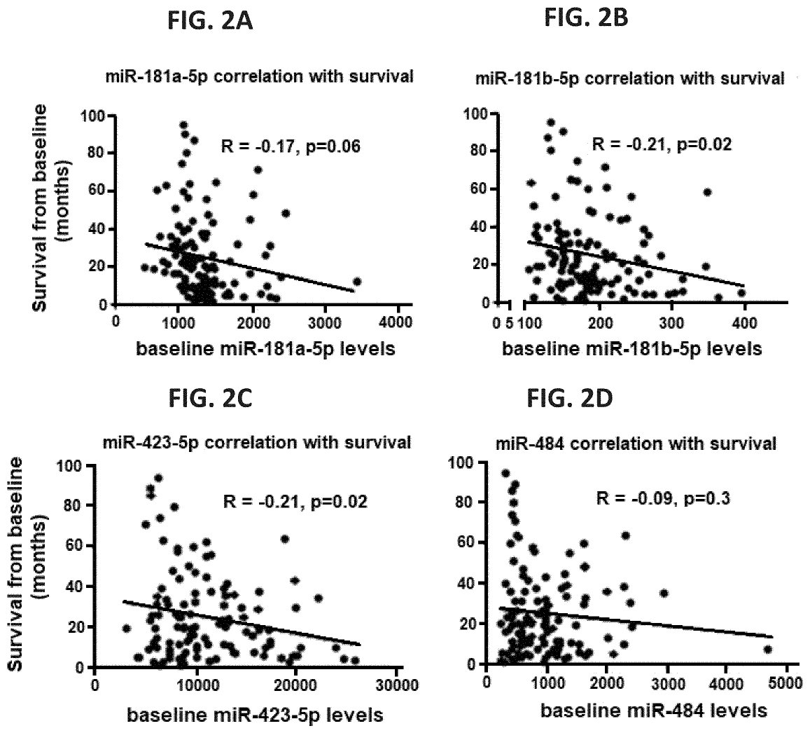

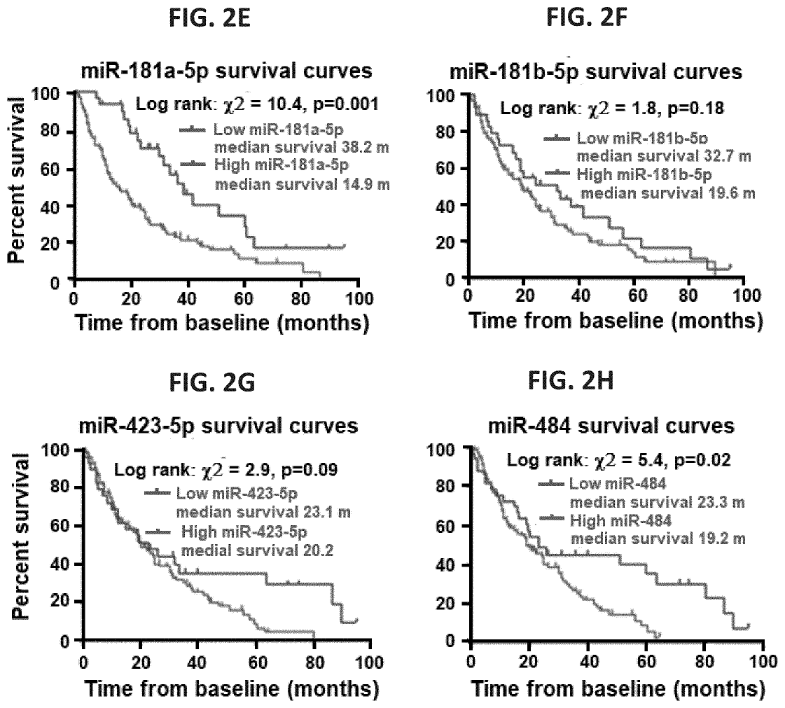

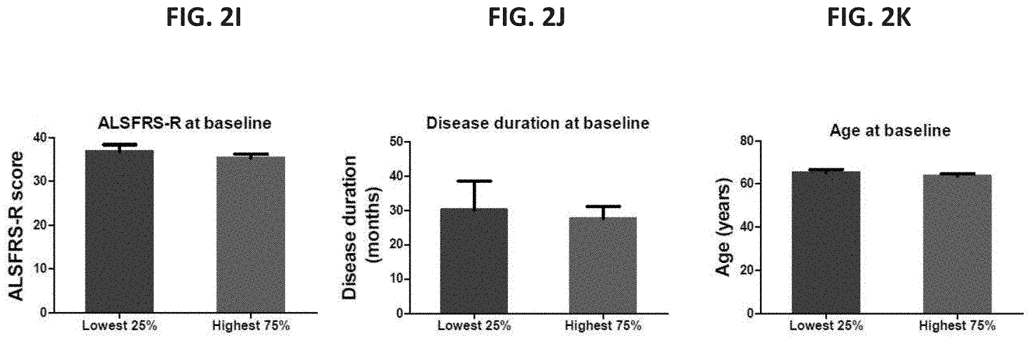

An unmet need in ALS clinical research is a prognostic biomarker that can predict disease course and/or survival. The present inventors therefore tested survival as a function of the expression of specific miRNA species. Survival length was calculated as the time in months from baseline (i.e., the first time patients were sampled, which was 28.5±3.4 months on average from symptom onset) to death. miR-181a-5p levels at baseline inversely correlated with survival length, approaching statistical significance (Pearson R=−0.17, p=0.06, FIG. 2A), while miR-181b-5p and miR-423-5p levels at baseline correlated significantly and inversely with survival length (miR-181b-5p: R=−0.21, p=0.02, FIG. 2B; miR-423-5p: R=−0.21, p=0.02, FIG. 2C, respectively). miR-484 levels did not significantly correlate with survival length (R=−0.09, p=0.3, FIG. 2D). miRNA levels were also correlated with disease duration, i.e. the survival time from symptom onset.

In a previous study it was demonstrated that NfL baseline levels can separate between cumulative survivals of ALS patient subgroups (Lu et al., 2015, supra]. Data analysis revealed that survival length could be stratified into subgroups by miR-181a-5p levels (log-rank Mantel-Cox: Chi-square=10.4, p<0.001, FIG. 2E), and less by miR-181b-5p (Chi-square=1.8, p=0.18, FIG. 2F). Stratification by miR-423-5p levels did not show a statistically significant difference (Chi-square=2.9, p=0.09, FIG. 2G), while miR-484 levels had a modest but significant effect (Chi-square=5.4, p=0.02, FIG. 2H). To exclude clinical status of patients as a confounding factor for miR-181a-5p levels, ALSFRS-R score at baseline (FIG. 2I), the time from symptom onset to baseline (FIG. 2J) and the age at baseline (FIG. 2K), were compared, between different quantiles of miR-181a-5p, and found no differences in either one (ALSFRS-R: p=0.16; disease duration: p=0.76; age: p=0.39;).

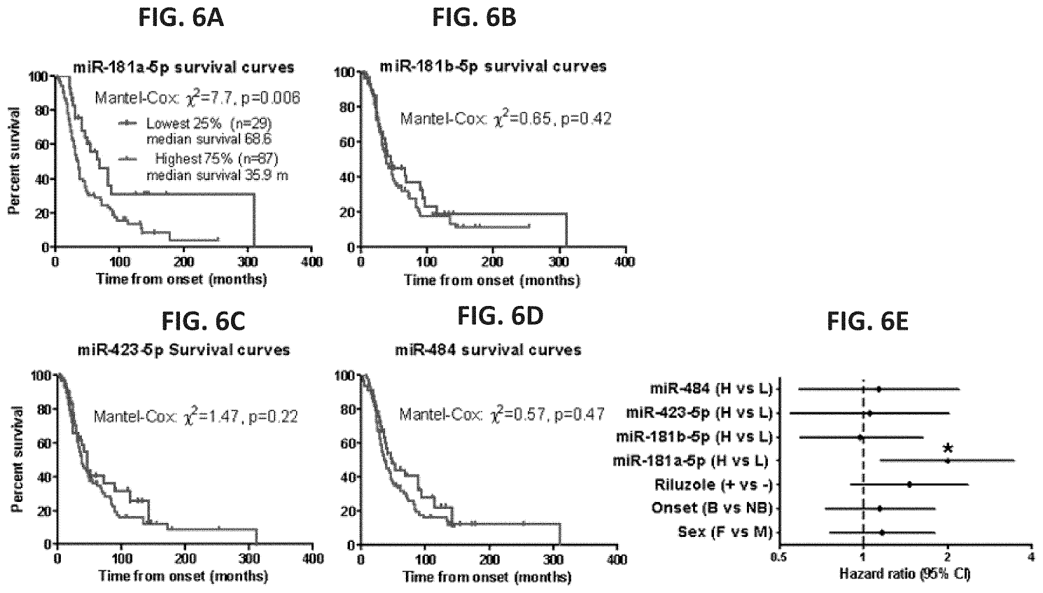

Log rank analysis for Kaplan-Meier curves was also performed using disease duration as defined above (FIGS. 6A-D). The findings for miR-181a-5p obtained using this approach were comparable to those in which survival was calculated from baseline, with higher levels of miR-181a-5p predicting poor survival (FIGS. 6A and 6E). Therefore, miR-181-5p levels were indicated as new cell-free molecular prognostic markers for ALS.

### Example 3

Mortality Rate Increases as a Function of miR-181-5p Levels

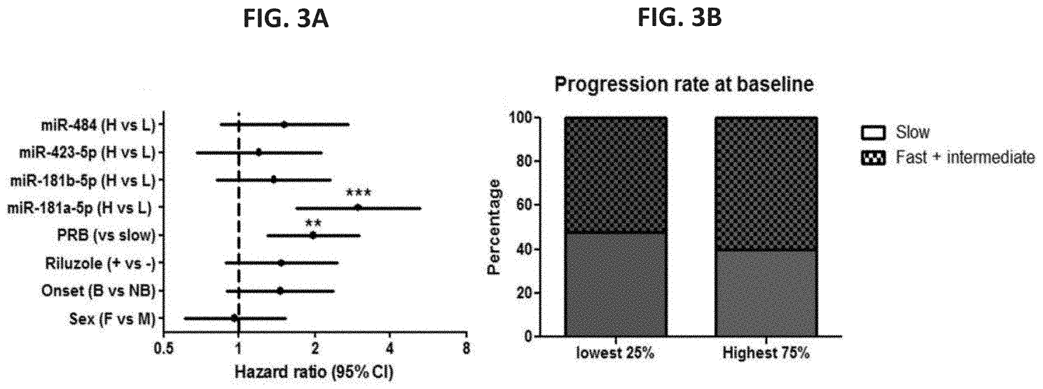

In order to further determine miRNA predictive power for death risk, multivariate Cox regression analysis was performed which yielded mortality hazard ratios (HRs), namely the ratio between the number of deaths per unit of time (mortality rates) of two patient populations. HR was statistically significant for miR-181a-5p (p<0.001) patients whose miR-181a-5p levels where within the top 75% quantile exhibiting a hazard ratio of approximately 3.1 relative to the lowest 25% (FIG. 3A). Therefore, high miR-181-5p levels predict an approximately tripled risk of death for ALS patients. The possibility that the effect of miR-181-5p is confounded by difference in progression rate was excluded, as the frequency of slow progressors vs. intermediate and fast progressors did not differ between the quantiles (Fisher's exact test: p>0.05, FIG. 3B). When survival was calculated from symptom onset rather than from baseline, HR was also significant for high levels of miR-181a-5p vs. low levels (2.0, p<0.05, FIG. 6E).

Taken together, higher miR-181a-5p plasma levels at baseline strongly associated with poor prognosis and increased mortality rates.

### Example 4

miR-423-5p and miR-484 Increase in Plasma as Disease Progresses

In addition, the present inventors tested a longitudinal cohort of 23 ALS patients that were phlebotomized repeatedly at four different time points (t₁-t₄). Average disease duration at t₁ (time from symptom onset) was 28.8±3.4 months; time to t₂ from t₁ was >3 months and on average 6.3±0.3 months; time to t₃ from t₁ was >9 months and on average 13.0±0.33 months; finally, time to t₄ from t₁ was >16 months and on average 32.7±3.3 months. Thus, disease duration at t₄ was 61.5±3.3 months, approximately twice as long than at t₁.

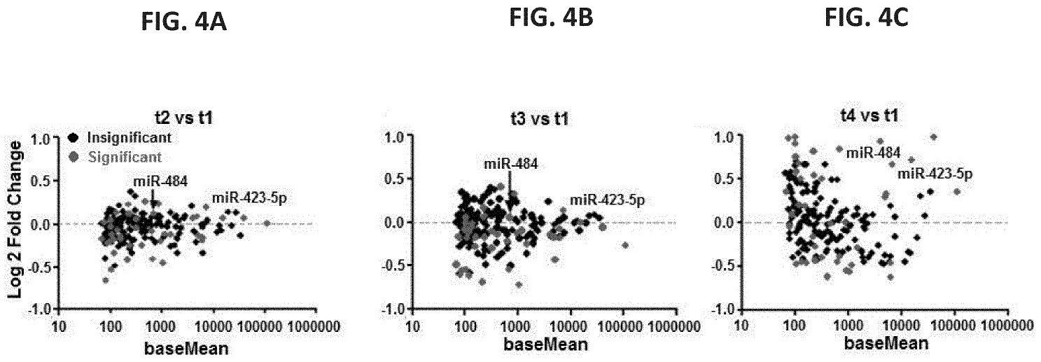

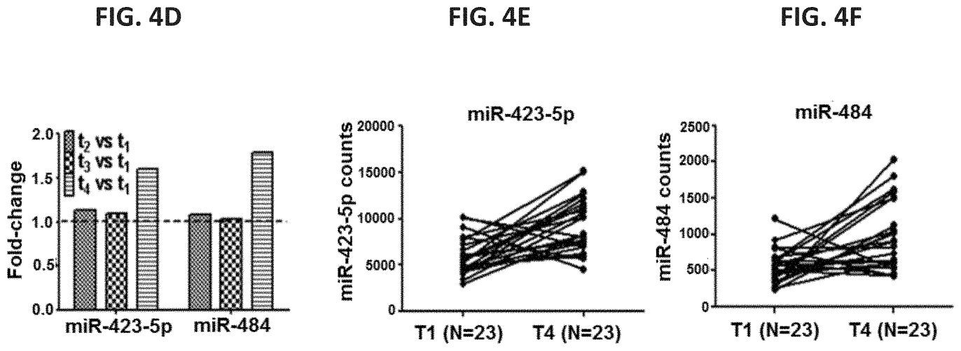

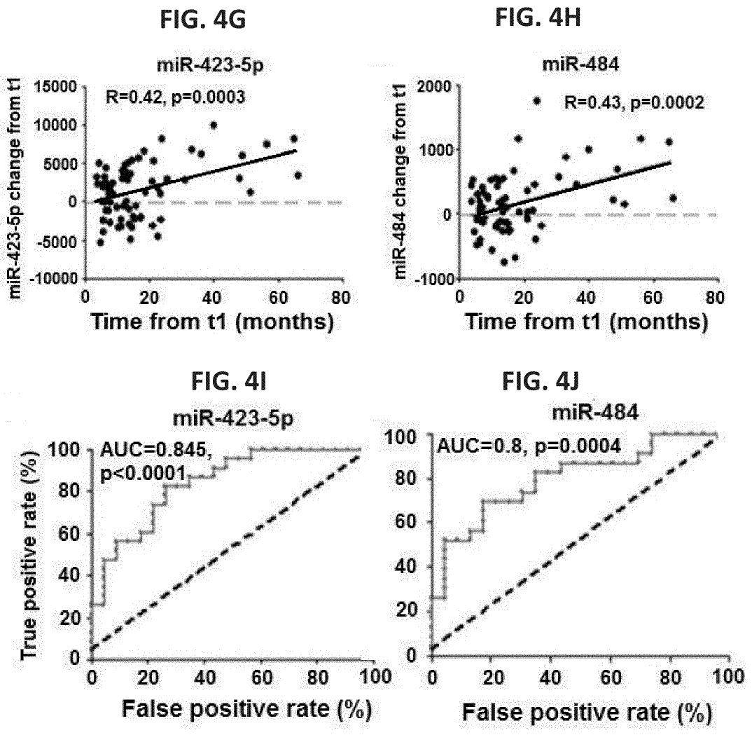

Measurements of miR-423-5p and miR-484 levels in t₂ and t₃, compared to baseline exhibited modest increase of approximately 1.15 in a statistically significant manner (p<0.001; FIGS. 4A-B, respectively). However, by the fourth sampling event (t₄), miR-423-5p levels increased by 1.6-fold compared to baseline levels, and miR-484 increased by 1.8-fold (FIGS. 4C-D). Noteworthy, both miR-423-5p and miR-484 showed only a very modest increase in ALS compared to control at baseline (miR-423-5p: fold-change=1.15, adjusted p-value=0.06; miR-484: fold-change=1.33, adjusted p-value=0.008), but a higher increase at t₄ (miR-423-5p: fold-change=1.5, adjusted p-value=0.004; miR-484: fold-change=2.23, adjusted p-value<0.001). The levels of miR-181a-5p and miR-181b-5p did not change longitudinally (data not shown).

Because the time intervals between sampling event were variable between patients, chronological analysis of changes in miR-423-5p and miR-484 levels were performed which were positive and with a highly significant correlation (miR-423-5p: R=0.42, p=0.0003, FIG. 4E; miR-484: R=0.43, p=0.0002; FIG. 4F). These data suggest that miR-423-5p and miR-484 levels increase linearly with time.

The predictive power of miR-181a-5p, miR-181b-5p, miR-423-5p and miR-484 were determined for classification of patient at t₄ vs. patients at t₁ by generating receiver operating characteristic (ROC) curves. Neither miR-181a-5p nor miR-181b-5p had a predictive power above the chance level of 0.5, with areas under the curve (AUCs) of 0.53±0.09 (p=0.71, FIG. 4G) and 0.51±0.09 (p=0.94, FIG. 4H), respectively. However, miR-423-5p and miR-484 had a high and statistically significant predictive power (miR-423-5p: AUC=0.845±0.056, p-value<0.0001, FIG. 4I; miR-484: AUC=0.803±0.065, p-value=0.0004, FIG. 4J), indicating that the levels of miR-423-5p and miR-484 can clearly differentiate between earlier and later measurements of the same patient.

Thus, miR-423-5p and miR-484 levels may be used as classifiers to distinguish between patients at different disease stages. One potential application of miR-423-5p and miR-484 is in assessing patient disease stage when enrolling into clinical trials. In addition, as both miRNAs increase longitudinally and in a linear manner, change in their dynamics might be considered a pharmacodynamic marker for clinical drug development.

### Example 5

miR-423-5p Levels Correlate with NfL and TNF-α Levels in Plasma

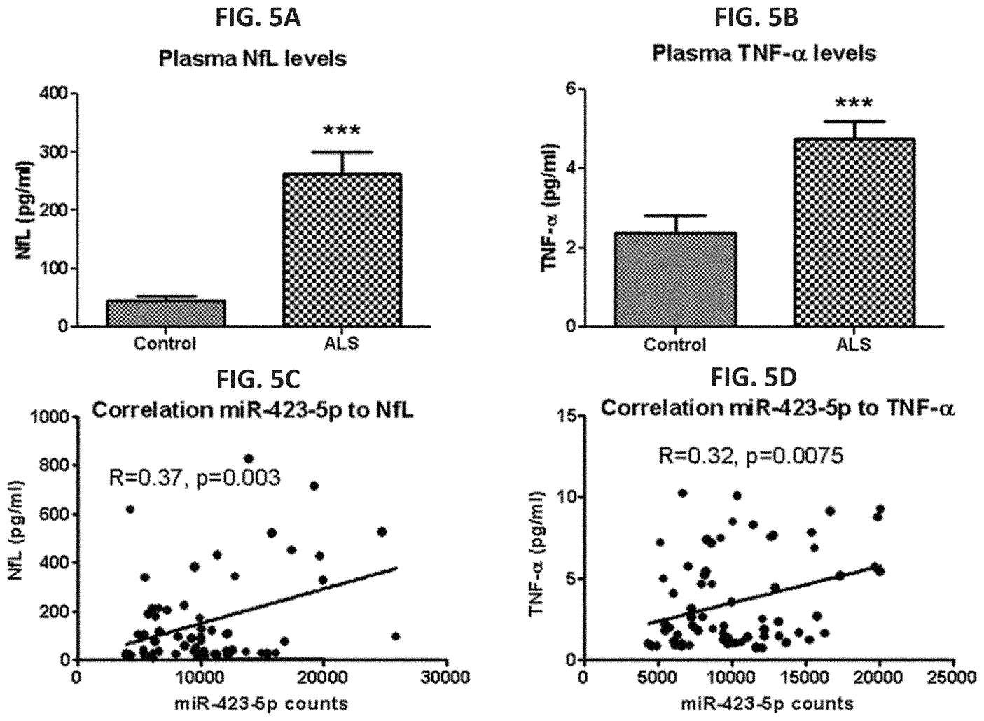

Because higher miR-423-5p levels were found to be associated with disease progression longitudinally, its association with neuronal damage and inflammatory markers was assessed. Plasma levels of NfL, a product of neuroaxonal breakdown [Lu et al., 2015, supra] and of TNF-α, a pro-inflammatory cytokine [Lu et al., 2016, supra] are known to be associated with ALS. Higher baseline NfL levels are associated with poor survival [Lu et al., 2015, supra], reminiscent of miR-423-5p. An almost six-fold increase in NfL levels was observed in a cohort of ALS patients (N=31) compared to a healthy control cohort (N=30; FIG. 5A, 262.9±38.4 pg/ml vs. 44.6±7.4 pg/ml, respectively, unpaired t-test: t(59)=5.5, p<0.0001). TNF-α levels increased by two-fold [FIG. 5B, 4.8±0.4 pg/ml in ALS (N=34) vs. 2.4±0.5 pg/ml in controls (N=35), unpaired t-test: t(67)=3.8, p=0.0003]. Plasma miR-423-5p levels at baseline correlated significantly with both NfL levels (Pearson: R=0.37, p=0.003, FIG. 5C) and TNF-α levels (R=0.32, p=0.0075, FIG. 5D).

Taken together, cell-free miR-181-5p, miR-423-5p and miR-484 levels are valuable and can be utilized to predict the progression of ALS and the mortality rate associated with it, and to contribute in the stratification of patient groups in clinical trials.

### Example 6

Further Evidence for the Use of miRNAs as Biomarkers for ALS

Unbiased next generation sequencing was used to investigate, without an a priori bias, the comprehensive landscape of plasma miRNAs in ALS patients with available clinical information (Table 2, below).

TABLE 2
Summary of demographic and clinical characteristics
of ALS cohorts I and II samples

| | Cohort I | | Cohort II | |
|---|---|---|---|---|
| Number of subjects (% males) | 115 | (64.3%) | 56 | (48.2%) |
| Age at enrolment | 64 ± 1 | yr. | 65 ± 2 | yr. |
| Age of onset (1^{st} reported | 61 ± 1 | yr. | 62 ± 2 | yr. |
| symptoms) | | | | |
| Disease duration at enrolment | 30 ± 3 | m. | 35 ± 6 | m. |

| | | |
|---|---|---|
| ALSFRS-R at enrolment | 36 ± 1 | 37 ± 1 |
| El-Escorial (Definite/probable/lab- | 34/51/15/15/0 | 3/14/21/15/3 |
| supported/possible/suspected) | | |
| Bulbar onset/total | 38/115 | 17/56 |
| Riluzole treated/total | 80/115 | 34/56 |
| ALS Family history/total | 7/115 | unknown |
| C9ORF72 genetics/total | 8/115 | 2/56 |

Abbreviations: ALSFRS-R: ALS functional rating scale, revised.

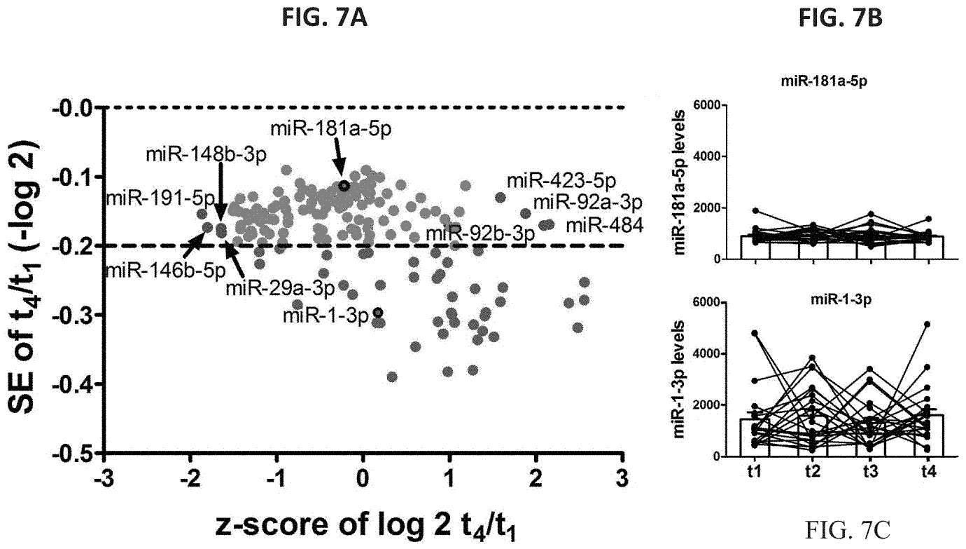

In the search for a prognostic biomarker that is stable throughout the disease course, miRNA measurement variability (noise) over time was experimentally assessed, by analyzing a longitudinal sample cohort of 22 patients, with four longitudinal blood samples taken: (t₁-t₄) during the course of 30 months (2.5 years). 88 samples were prepared from total plasma RNA, as previously described [Coenen-Stass et al., 2018, supra], and profiled by small RNA next generation sequencing (RNA-seq). Linear miRNA quantification was achieved via unique 12-nucleotide molecular identifiers (UMIs). miRNAs with ≥50 UMIs in at least 60 percent (N=53) of the samples were considered above noise level. Thus, of 2008 miRNAs aligned to the human genome (GRCh37/hg19), 179 passed the threshold which was set (data not shown). Measured variability between 22 individuals was low for 126/179 miRNAs (−log 2 of t₄/t₁ standard error≥−0.2), and these were considered stable, while 53/179 other miRNAs exhibited higher variability between patients and were excluded from further analysis (FIG. 7A, y-axis). For example, miR-181a-5p levels exhibited reduced variability at each time point across individual patients, in comparison with miR-1-3p (FIGS. 7B-C).

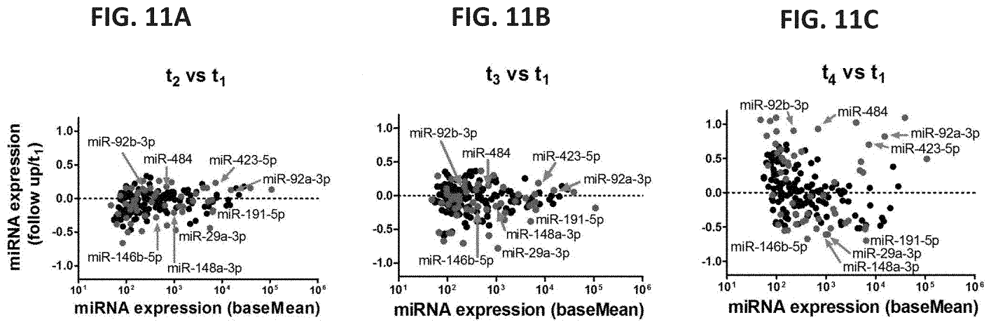

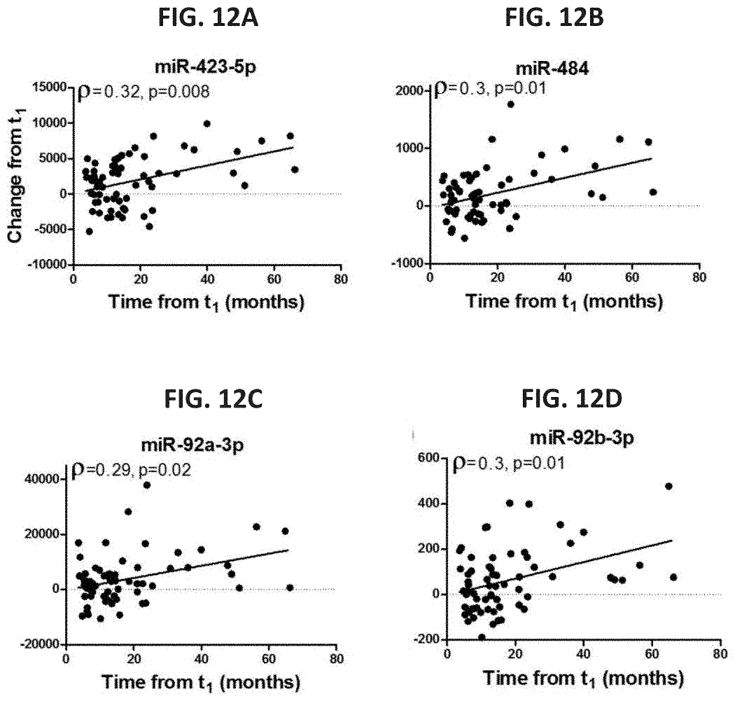

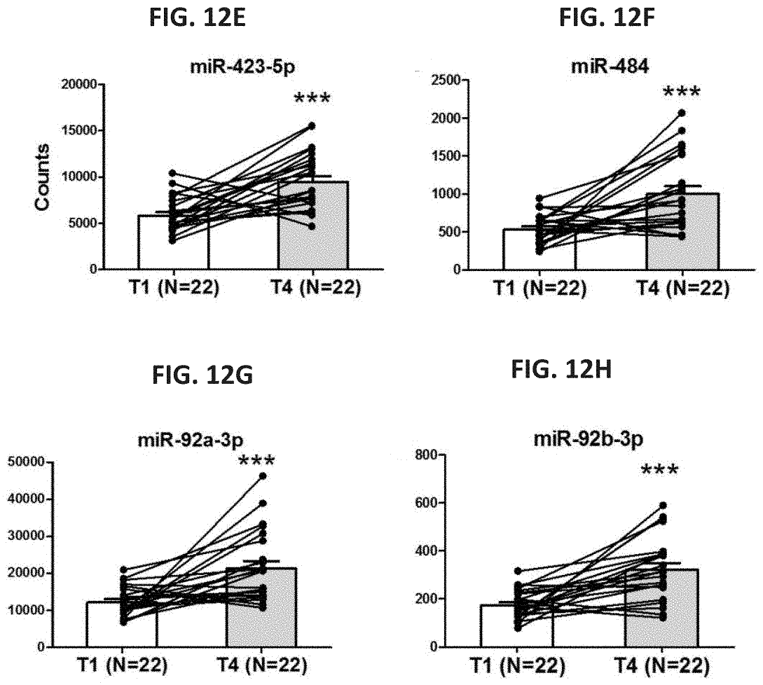

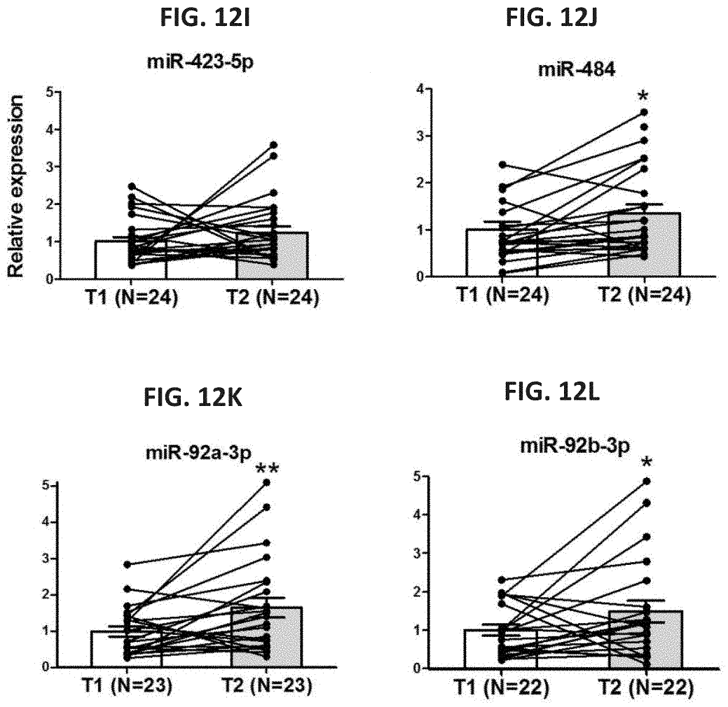

miRNAs could be further characterized by intra-personal changes over time. Intriguingly, four miRNAs (miR-423/484/92a/b) exhibited a robust increase in abundance from t₁ to t₄ (FIG. 7A, x-axis; Z-score of log 2 t₄/t₁>1.5 SD), while four other miRNAs (miR-29/146/148/191) exhibited a decrease (Z-score of log 2 t₄/t₁<−1.5 SD). miR-423/484/92a/b modestly increased early on (approximately 1.15 fold t₂ or t₃ relative to t₁), and further increased approximately 1.7-fold upon the fourth sampling (t₄/t₁), while miR-29/146/148/191 decreased by up to 1.6-fold upon the fourth sampling (FIGS. 11A-C). The correlation of miR-423/484/92a/b levels to disease progression was also observed when time from enrolment (in months) was considered as a continuous variable (FIGS. 12A-D), while those of miR-29/146/148/191 showed a less significant correlation (ISpearman rhol<0.2, p>0.05; not shown). Individual trajectories of miR-423/484/92a/b exhibited increases in most of the patients from t₁ to t₄ (FIGS. 12E-H). Furthermore, these miRNAs increased significantly in a replication longitudinal cohort (N=22-24 patients, one-tailed t-test: miR-484, p=0.01; miR-92a-3p, p=0.006; miR-92b-3p, p=0.04; FIGS. 12J-L), whereas the results for miR-423-5p where less significant in the second longitudinal cohort (one-tailed p-value=0.16, FIG. 12I).

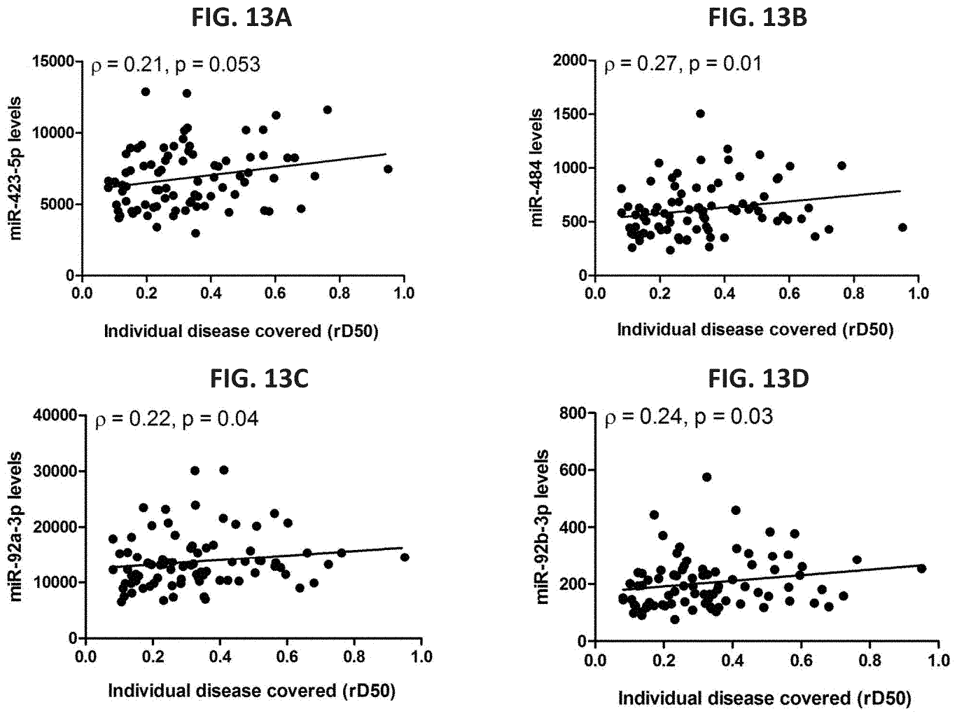

The relative D50 (rD50), a modelled derivative of ALSFRS-R decay, reveals the disease covered by individual patients independent of the rate of progression [Prell et al., 2019, supra]. For example, an rD50 of 0.0 and 0.5 signify ALS onset and the time-point of halved functionality, respectively [Prell et al., 2019, supra]. miR-484/92a/b levels measured at longitudinal blood samplings from the same patients correlated with rD50 at the time of sampling (FIGS. 13B-D) and may be considered as candidate molecular biomarkers which increase in all patients independent of individual disease aggressiveness.

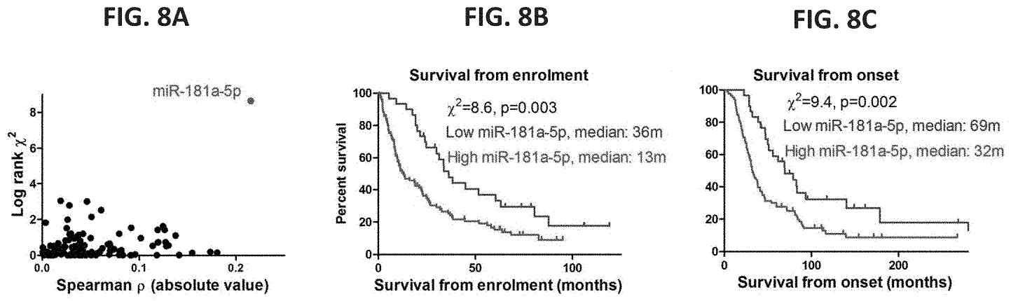

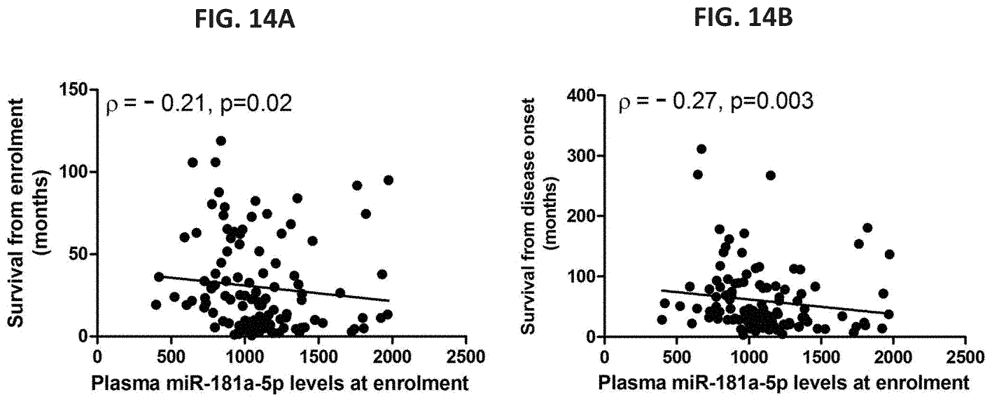

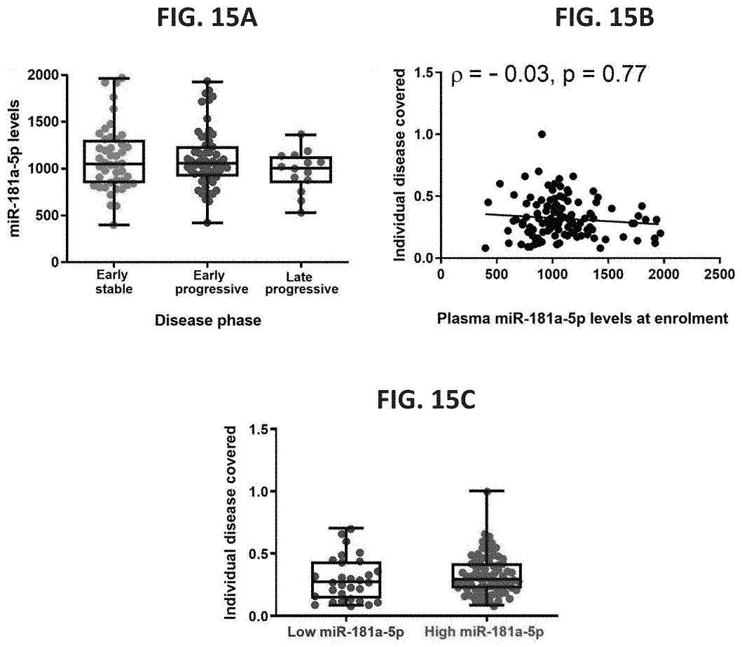

The potential blood-borne miRNAs for prognostic biomarkers was further explored. An ideal prognostic biomarker should be preferentially stable throughout the course of disease. 118 miRNA candidate biomarkers, whose plasma levels were relatively stable during disease progression were identified (green features in FIG. 7A). miRNA expression at enrolment was then correlated to survival duration, calculated as lifespan from either symptom onset or from first phlebotomy at study enrolment (on average 30.0±6 months after symptom onset). The levels of one miRNA, miR-181a-5p exhibited significant correlation to survival length (Spearman correlation coefficient=−0.21, p=0.02, FIGS. 8A and 14A). Because miR-181a-5p levels remain stable in time (FIGS. 1A-B), it is unlikely that measurements are confounded by sampling at different disease stages. The stability of miR-181a-5p during disease progression was also indirectly implied by comparable miR-181a-5p levels between patients sampled at different (early stable, early progressive and late progressive) disease phases (0≤rD50<0.25; 0.25≤rD50<0.5; rD50≥0.5, respectively; ANOVA p=0.35, FIG. 15A) and by lack of correlation between miR-181a-5p levels and rD50 at enrolment (FIG. 15B).

Patients were further subdivided in quartiles according to the expression level of individual miRNA genes, and the low quartile (30 patients) were compared to the rest of the cases (85 patients). Differences in plasma miR-181a-5p levels were able to predict survival length better than any other miRNA. Kaplan Meier curves revealed clear separation of survival between the subgroups, based on plasma miR-181a-5p levels at enrolment (log-rank test chi-square=8.6, p=0.003, FIG. 8B). The median patient survival in the lower miR-181a-5p quartile was 36 months, compared to 13 months at the higher three quartiles. Thus, plasma miR-181a levels predict a substantial median survival difference of 23 months that is equivalent to a 280% increase in survival length for patients with lower plasma miR-181a-5p levels. Comparable results were gained, when plasma miR-181a-5p levels at enrolment were stratified against survival from reported first symptoms (disease onset, log-rank chi-square=9.4, p=0.002; FIG. 8C).

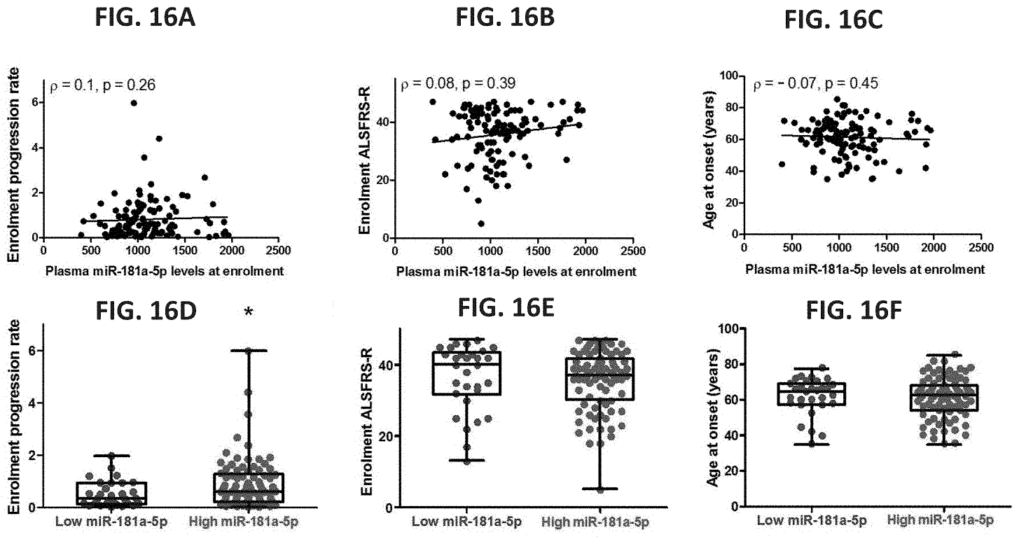

It is noteworthy that patient individual disease covered at enrolment (rD50) was comparable between the low quartile of miR-181a-5p and the rest of the cases (FIG. 15C). Furthermore, enrolment progression rate, enrolment ALSFRS-R scores, and chronological age at onset were not correlated with miR-181a-5p levels, and these phenotypic features did not differ between the low and high miR-181a-5p quantiles, except for a significant but modest increase in enrolment progression rate (FIG. 16A-F). These findings exclude the possibility that survival differences between miR-181a-5p expression bins result from factors other than miR-181a-5p expression levels, such as the clinical status of the patients when they are sampled.

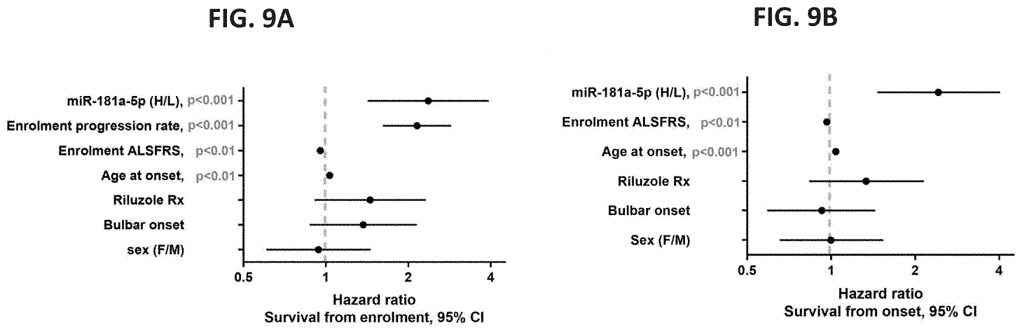

The monthly mortality hazard ratio (HR) was calculated for different plasma miR-181a-5p quantiles and clinical covariates, by multivariate Cox regression analysis. This analysis allows calculation of an independent hazard ratio for each covariate while holding the other covariates constant. High miR-181a-5p levels (top three quartiles) exhibited a hazard ratio of 2.35 (95% CI 1.4-3.9), relative to the lowest quartile when calculated from enrolment (Wald test p<0.001, FIG. 9A), and a hazard ratio of 2.4 when calculated from disease onset (95% CI, 1.5-4.0, p<0.01, FIG. 9B). Enrolment progression rate, i.e. the rate of change in ALSFRS from symptom onset to enrolment, was also associated with a high hazard ratio of 2.16 for survival time from enrolment (95% CI 1.6-2.9, p<0.001, FIG. 9A), as previously reported [Elamin et al. J Neurol (2015) 262: 1447-1454]. Taken together, higher miR-181a plasma levels at enrolment are associated with a considerably poor prognosis in ALS, an effect which is not confounded by other phenotypic properties.

### Example 7

Validation of miRNA-181 as a Prognostic Marker in a Second Cohort Group

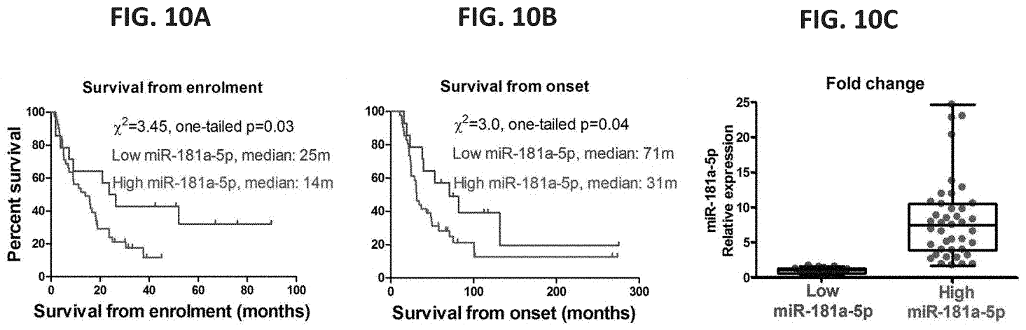

In order to validate the above described findings, a replication study was performed by measuring miR-181a-5p levels in plasma of an independent cohort of 56 patients (see Table 2, above) by an orthogonal qPCR methodology. The replication study confirmed the existence of a prognostic gap of 180%, i.e. 1.8-fold increase in survival, from 14 to 25 months, between patients with high or low plasma miR-181a-5p levels, respectively (Log rank test: chi square=3.45, one-tailed p-value=0.03, FIG. 10A). The survival difference was reproduced in the replication cohort, even when calculated from first reported symptoms (230%, 31 months from disease onset to 71 months, chi square=3.0, one-tailed p-value=0.04, FIG. 10B). Importantly, the average miR-181a-5p levels varied by 8-fold between the plasma of patients with better prognosis, relative to those with poor prognosis (FIG. 10C), suggesting a good dynamic range for biomarker quantification. Thus, the data from the two different cohorts of ALS patients established miR-181-5p as a new cell-free prognostic biomarker for ALS.

### Example 8

Prognostic Value of miR-181-5p and NfL

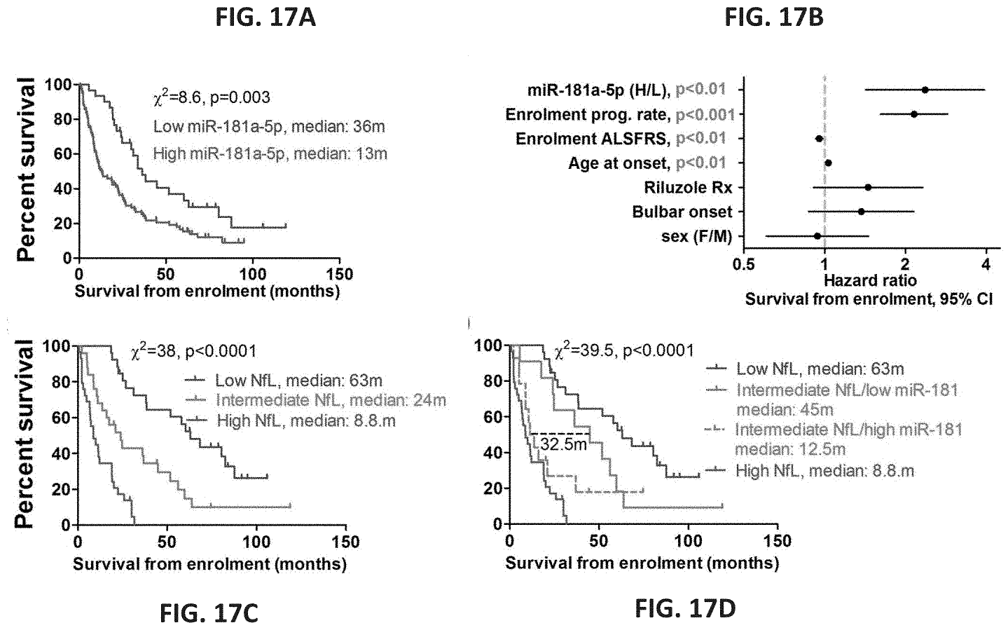

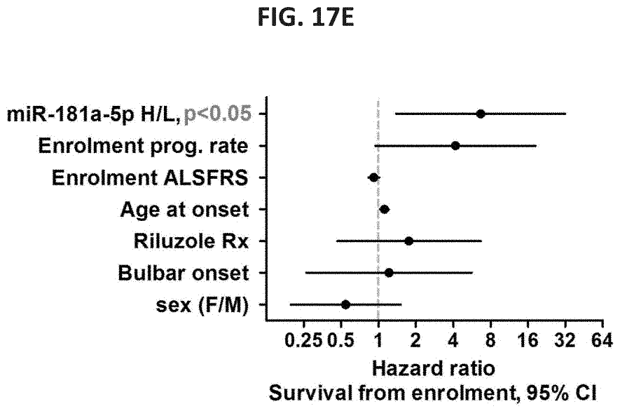

The present inventors further tested the prognostic value of miR-181 in the context of neurofilament light chain (NfL), the benchmark prognostic marker in experimental setting. Utilizing 80 out of the 115 samples which had NfL measurements, it was uncovered that while NfL levels stratified patients by survival length, as previously shown (Chi-square=38, p<0.0001, FIG. 17C), miR-181 levels could further sub-stratify patients with intermediate NfL levels, with a gap of 32.5 months in survival length between low and high miR-181 levels, equivalent to 3.6-fold increase in low miR-181 (Chi-square=39.5, p<0.0001, FIG. 17D). High miR-181 levels predicted a HR of ×6.6 (p<0.05) compared to low miR-181 levels within this subgroup (FIG. 17E).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

1. A method of prognosing a course of disease progression and/or survival time in a subject diagnosed with Amyotrophic lateral sclerosis (ALS) or Frontotemporal dementia (FTD), the method comprising:
(a) detecting a level of miR-181 in a biological sample of the subject; and
(b) determining the disease progression and/or survival time based on said level of said miR-181, wherein:
(i) when said level of miR-181 is higher than that in a control sample, it is indicative of a rapid disease progression and/or poor survival; or
(ii) when said level of miR-181 is about the same or lower than that in said control sample, it is indicative of a slow disease progression and/or good survival,
thereby prognosing the course of disease progression and/or survival time.

2. A method of treating ALS or FTD in a subject in need thereof, the method comprising:
(a) prognosing the subject according to the method of claim 1; and
(b) treating the subject based on the prognosis, wherein:
(i) when the prognosis indicates a rapid disease progression and/or poor survival, the subject is treated with an effective amount of a drug or a medicament for the treatment of rapid progressing disease; or
(ii) when the prognosis indicates a slow disease progression and/or good survival, the subject is treated with an effective amount of a drug or a medicament for the treatment of slow progressing disease,
thereby treating the ALS or FTD in the subject.

3. A method of prognosing a stage of disease in a subject diagnosed with ALS or FTD, the method comprising:
(a) detecting at least two times in a course of said disease a level of miRNA of at least one of miR-423, miR-484 and miR-92 in a biological sample of the subject; and
(b) determining a stage of said disease based on said level of miRNA, wherein an increase in said level of miRNA over said at least two times in said course of said disease is indicative of progression of said disease,
thereby prognosing the stage of disease.

4. A method of prognosing a stage of disease in a subject diagnosed with ALS or FTD, the method comprising:
(a) detecting at least two times in a course of said disease a level of miRNA of at least one of miR-29, miR-146, miR-148 or miR-191 in a biological sample of the subject; and
(b) determining a stage of said disease based on said level of miRNA, wherein a decrease in said level of miRNA over said at least two times in said course of said disease is indicative of progression of said disease,
thereby prognosing the stage of disease.

5. A method of treating ALS or FTD in a subject in need thereof, the method comprising:
(a) prognosing the subject according to the method of claim 3; and
(b) treating the subject based on the prognosis, wherein:
(i) when the prognosis indicates an early stage of disease, the subject is treated with at least one of an effective amount of drug or medicament and/or an assistive device;
(ii) when the prognosis indicates a middle stage of disease, the subject is treated with at least one of an effective amount of a drug or medicament, a physical therapy, an assistive device, a feeding tube, and/or a noninvasive ventilation; or
(iii) when the prognosis indicates a late stage of disease, the subject is treated with at least one of an effective amount of a drug or medicament, a physical therapy, an assistive device, a feeding tube, and/or a noninvasive or invasive ventilation,
thereby treating the ALS or FTD in the subject.

6. A method of monitoring treatment in a subject diagnosed with ALS or FTD, the method comprising:
(a) treating a subject diagnosed with ALS or FTD with a drug or a medicament;
(b) detecting a level of miRNA of at least one of miR-423, miR-484, miR-92, miR-29, miR-146, miR-148 or miR-191 in a biological sample of the subject prior to and following said treatment; and
(c) determining an effective treatment based on said level of miRNA, wherein:
(i) when said level of said miR-423, miR-484 and/or miR-92 is about the same or lower than that in a sample of the subject prior said treatment, it is indicative of an effective treatment; or
(ii) when said level of said miR-29, miR-146, miR-148 and/or miR-191 is about the same or higher than that in a sample of the subject prior said treatment, it is indicative of an effective treatment;
thereby monitoring treatment of the drug or the medicament for the treatment of ALS or FTD.

7. A method for selecting subjects for enrollment in a clinical trial involving treatment of ALS or FTD, the method comprising:
(a) detecting a level of miR-181 in a biological sample of a subject diagnosed with ALS or FTD;
(b) determining the disease progression and/or survival time based on said level of said miR-181, wherein:
(i) when said level of miR-181 is higher than that in a control sample, it is indicative of a rapid disease progression and/or poor survival; or
(ii) when said level of miR-181 is about the same or lower than that in said control sample, it is indicative of a slow disease progression and/or good survival;
and
(c) identifying the subject as being suitable for said clinical trial based on the criteria of said clinical trial.

8. A method for selecting subjects for enrollment in a clinical trial involving treatment of ALS or FTD, the method comprising:
(a) detecting at least two times in a course of a disease a level of miRNA of at least one of miR-423, miR-484 and miR-92 in a biological sample of a subject diagnosed with ALS or FTD;
(b) determining a stage of said disease based on said level of miRNA, wherein an increase in said level of miRNA over said at least two times in said course of said disease is indicative of progression of said disease; and
(c) identifying the subject as being suitable for said clinical trial based on the criteria of said clinical trial.

9. A method for selecting subjects for enrollment in a clinical trial involving treatment of ALS or FTD, the method comprising:
(a) detecting at least two times in a course of a disease a level of miRNA of at least one of miR-29, miR-146, miR-148 or miR-191 in a biological sample of a subject diagnosed with ALS or FTD;
(b) determining a stage of said disease based on said level of miRNA, wherein a decrease in said level of miRNA over said at least two times in said course of said disease is indicative of progression of said disease; and
(c) identifying the subject as being suitable for said clinical trial based on the criteria of said clinical trial.

10. The method of claim 3, wherein one of said at least two times in said course of said disease comprises a biological sample obtained at disease onset or at time of diagnosis.

11. The method of claim 2, wherein said drug comprises Riluzole, or Edavarone.

12. The method of claim 1, further comprising collecting said biological sample from the subject.

13. The method of claim 1, wherein said biological sample is cell-free.

14. (canceled)

15. The method of claim 1, wherein said miR-181 is a cell-free miRNA.

16-17. (canceled)

18. The method of claim 1, wherein said higher level of said miR-181 is by at least about 50%.

19. The method of claim 1, wherein said lower level of said miR-181 is by about 5-30%.

20. The method of claim 1, wherein said miR-181 is miR-181a-5p.

21. The method of claim 1, wherein said miR-181 is miR-181b-5p.

22. The method of claim 1, wherein said determining does not comprise a ratio of said miR-181 to a second miRNA selected from the group consisting of let-7e, miR-7, miR-9, miR-9*, miR-16, miR-29a, miR-31, miR-99b, miR-125b, miR-128a, miR-129-3p, miR-138, miR-155, miR-204, miR-218, miR-323-3p, miR-335, miR-338-3p, miR-451, miR-491 and miR-874.

23. The method of claim 3, wherein an increase in said level of said miRNA is by at least about 50%.

24-26. (canceled)

27. The method of claim 4, wherein a decrease in said level of said miRNA is by at least 50%.

28-31. (canceled)

32. The method of claim 4, wherein said determining does not comprise a ratio of said miR-29 to a second miRNA selected from the group consisting of miR-7, miR-9*, miR-99b, miR-181a, miR-206 and miR-335.

33. The method of claim 1, further comprising assessing a level of a neurofilament light chain (NfL) in said biological sample.

34. The method of claim 1, further comprising assessing a level of at least one pro-inflammatory cytokine in said biological sample.

35. The method of claim 1, wherein the subject is a human being.
